(19) United States
(12) Patent Application Publication
(10) Pub. No.: US 2026/0275414 A1
(43) Pub. Date: Sep. 17, 2026

(54) MODULATION OF TARGET MOLECULE-LIPID BILAYER INTERACTIONS

(71) Applicant: Roche Sequencing Solutions, Inc., Pleasanton, CA (US)

(72) Inventors: Aisha LAGUERRE, Sunnyvale, CA (US); Courtney NGUYEN, Morgan Hill, CA (US); Rongxin NIE, San Jose, CA (US); Adolfo VARGAS, San Jose, CA (US)

(21) Appl. No.: 19/469,083

(22) PCT Filed: Mar. 21, 2024
(86) PCT No.: PCT/EP2024/057565

§ 371 Date: Sep. 25, 2025

Related U.S. Application Data
(60) Provisional application No. 63/455,851, filed on Mar. 30, 2023.

(51) Int. Cl. C12Q 1/6869; G01N 33/487

(52) U.S. Cl. CPC C12Q 1/6869; G01N 33/48721

(57) ABSTRACT

Provided are lipid binding molecules and/or combinations of the lipid binding protein with a lipid component (i.e., a mispid) that are used to modify the interaction of a target molecule with a lipid membrane. This includes use of the lipid binding molecules and/or mispids, for example, to improve sequencing efficiency and throughput of nanopore-based sequencing systems. To sequence a target molecule, such as a nucleic acid sequence or a surrogate nucleic acid polymer derived therefrom, lipid binding molecules and/or mispids thereof are combined with the target molecule. The mixture is then applied to a nanopore-based sequencing chip. The target molecule is then sequenced in the presence of the lipid binding molecules and/or nanodiscs, thereby improving the capture, arrival time, and effective concentration of the target molecule across the membrane of the chip. Such improved efficiency is particularly beneficial, for example, when concentrations of a target molecule are low.

### STATEMENT REGARDING SEQUENCE LISTING

The Sequence Listing associated with this application is provided in xml format in lieu of a paper copy, and is hereby incorporated by reference into the specification. The name of the text file containing the Sequence Listing is P37738-WO_Sequence_Listing.xml. The xml file is 6500 bytes, and was created on Mar. 13, 2024.

### FIELD OF THE INVENTION

The present invention relates generally to modulating interactions between a target molecule and a lipid membrane, including modifying the interaction between a target molecule and the lipid membrane of a nanopore-based sequencing chip to improve sequencing efficiency and throughput.

### BACKGROUND

Over the last two decades, biological membranes have emerged as an important tool in a variety of biomedical applications. This includes the use of lipid membranes in nanopore-based sequencing applications, where nanopores provide as a constant and reproducible physical aperture, through which a target molecule can be directed and sequenced.

Generally, nanopores are associated with a chip-based flow cell, the flow cell having an inlet to which a solution including the target molecules is added. The flow cells also include multiples wells, each well having a lipid bilayer membrane and a single nanopore that is embedded within the membrane. Also associated with each well is a sensing electrode that can detect changes in current and/or voltage across the membrane of the well.

As the solution including a target molecule is introduced into the flow cell sample port, the solution flows across the lipid bilayer membranes of the wells, allowing nanopores within the membrane to capture the target molecules. The membrane, which exhibits a high electric resistance, allows ions to pass through the pore when a suitable potential is applied across the membrane, generating a current. The ionic current through the nanopore also drives a target molecule through the nanopore. As the target molecule passes through the nanopore, it causes changes in the ionic current, for example, which can be detected via the sensing electrodes associated with the wells of the flow cells. Each base of a nucleic acid that passes through the nanopore, for example, can then be identified through the characteristic disruption it causes to the current in real-time.

One challenge when using such biological, nanopore-based sequencing platforms is inefficiencies associated with the evenness of distribution of target molecules across the biological membrane of the chip (as the target molecules flow across the membrane). This is particularly true for target molecules having an affinity for the lipid bilayer membranes, as the solution including such target molecules may be depleted of the target molecule molecules—early in their flow across the flow cell—as the target molecules initially encounter the wells of the flow cell. These inefficiencies are additionally problematic when initial amounts of the target molecule are low, such as when the starting concentration of a DNA sample is low.

There is thus a need for improved flow of target molecules over the lipid bilayer membrane of a nanopore-based sequencing chip. In particular, there is a need for methods and systems that improve the arrival rate of target molecules to the nanopores and the uniformity of their capture by the nanopores. There is also a need for improving nanopore-based sequencing when initial concentrations of a target molecule are low. There is also more broadly a need for modifying the interaction of a target molecule with a lipid membrane generally, such as to modulate nanopore kinetics and/or to deliver molecules (such as metabolites) that can be detected by modulating pore behavior.

### SUMMARY OF THE INVENTION

In certain example aspects, provided is a composition for modifying the interaction of a target molecule with a lipid membrane. The composition includes, for example, a heterogenous mixture of lipids and lipid binding proteins, the lipid and lipid binding proteins forming stable complexes (i.e., “mispids”) of variable size and stoichiometries. In certain example aspects, the lipid binding protein is a membrane scaffolding protein (MSP), such as apolipoprotein (e.g., Apo-A1) or derivative thereof. Further, the lipid binding protein can include a predetermined number of alpha helices, such as 3-10 alpha helices. Further, in certain example aspects the ratio of lipid binding protein to lipid component is about 1:200 to 1:1500, such as 1:200.

In certain example aspects, provided are methods of modifying an interaction of a target molecule with a lipid bilayer. The methods include, for example, providing a lipid membrane and contacting the lipid membrane with multiple mispids, such as a solution of mispids and multiple target molecules, such as a solution of target molecules. The target molecules, for example, can have an affinity for the lipid membrane, but contacting the lipid membrane with the mispids reduces the affinity of the target molecule for the lipid membrane. As such, the interaction of the target molecule with the lipid membrane is modified via the use of the mispids.

In further example aspects, provided is a method for sequencing a target molecule. The method includes, for example, providing a chip, the chip including multiple wells, each well including a sensing electrode, a lipid membrane that is disposed adjacent to or in proximity to the sensing electrode, and a nanopore disposed within the lipid membrane. The chip is contacted, with multiple mispids, such as a solution of mispids. The chip is also contacts with multiple target molecules, such as target molecules that each have an affinity for the lipid membrane. A volage is applied across the membrane of the chip and, via one or more of the sensing electrodes, a current or voltage change associated with the nanopore is determined. A sequence for the target molecule is then determined, such as with the aid of a computer processor and based on the one or more of the determined current or voltage changes associated with the nanopore.

In still further example aspects, provided is a method sequencing a target molecule that optionally includes mispids. Such methods include, for example, providing a chip that includes multiple sensing electrodes and a lipid membrane that is disposed adjacent to or in proximity to the sensing electrodes. Multiple nanopore assemblies, for example, are also disposed within the lipid membrane. The chip is contacted with multiple lipid biding proteins and optionally multiple mispids. For example, the chip can be contacted with the lipid biding proteins alone. Nonetheless, the chip is also contacted with multiple target molecules, such as a solution including the target molecules. In certain example aspects, each target molecule has an affinity for the lipid membrane. A volage is applied accords the membrane of the chip, and one or more current or voltage changes associated with the nanopore assembly is determined, such as via one or more of the sensing electrodes associated with the chip. A sequence of the target molecule is then determined, such as with the aid of a computer processor and based on the one or more of the determined current or voltage changes associated with the nanopore assembly. In certain example aspects, at least a portion of the lipid binding proteins are complexed with a lipid component to form multiple mispids and the chip is contacted with the mispids.

In certain example aspects, the target molecule is a nucleic acid, a modified nucleic acid, or a nucleic acid surrogate. The nucleic acid surrogate, for example, can include an Xpandomer.

In certain example aspects, the lipid binding protein is a membrane scaffolding protein (MSP). For example, the MSP is an apolipoprotein A1 or derivative thereof. In certain example aspects, the lipid binding protein includes a predetermined number of alpha helices, such as such as 3, 4, 5, 6, 7, 8, 9 or 10 alpha helices.

In certain example aspects, the ratio of the lipid binding proteins to the lipid component is approximately 1:200. In certain example aspects, the ratio of the lipid binding protein to the lipid component is approximately 1:200 to 1:1500. For example, the ratio of the lipid binding protein to the lipid component is approximately 1:1200. In certain example aspects the ratio of the lipid binding protein to the lipid component is approximately 1:200.

In certain example aspects, the lipid component includes a phosphatidylcholine-based lipid, a phosphoethanolamine-based lipid, a derivative thereof, or a combination thereof. For example, the lipid component can include 1,2-Diphytanoyl-sn-Glycero-3-Phosphatidylcholine (DPhPC), 1,2-diphytanoyl-sn-glycero-3-phosphoethanolamine (DphPE), derivatives thereof, or combinations thereof.

In certain example aspects, the mispids and target molecules are mixed together before the chip or the membrane is contacted with the mispids and target molecules. In still other example aspects, the membrane or chip is contacted sequentially with the mispids and the target molecules or the target molecules and the mispids.

In certain example aspects, contacting the chip or the membrane with mispids and target molecules improves throughput associated with a sequencing reaction associated with the target molecule. In still further example aspects, contacting the chip or the membrane with the mispids and target molecules improves flow cell capture of the target molecule in a sequencing reaction associated with the target molecule.

In certain example aspects, each of the methods described herein can use nanodiscs or, in certain example aspects, a mixture of nanodiscs and mispids, or, in certain example aspects, mixtures of lipid binding proteins, mispids, and/or nanodiscs. For example, a membrane or chip, such as membrane associated with a sequencing chip, can be contacted with the lipid binding proteins described herein, the mispids described herein, nanodiscs as described herein, or a combination thereof. Such contacting the membrane and/or chip, for example, modifies the interaction of a target molecule with the membrane and/or chip. For example, when the target molecule has an affinity to a membrane, the contacting of the membrane lipid binding proteins, mispids, nanodiscs or combinations thereof can reduce the interaction of the target molecule with the membrane.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

FIG. 1A is an illustration depicting flow of target molecules across a lipid membrane of a flow cell well, in the absence of mispids, in accordance with certain example embodiments. As shown, when target molecules flow across a flow cell well, they bind to and accumulate on the lipid bilayer membrane of the well. Target molecules are thus depleted from the sample, leaving less target molecules to flow across the flow cell. Pore capture is in turn limited by how fast the pore can pull target molecules from bilayer in its lifetime.

FIG. 1B is an illustration depicting flow of target molecules across a lipid membrane of a flow cell well in the presence of the in the presence of mispids, in accordance with certain example embodiments. As shown, the mispids compete for space on the lipid bilayer of the flow cell well membrane. This reduces target molecule binding to the lipid membrane, thus freeing more target molecules to flow across the flow cell generally (and to additional wells in this example). This in turn improves confluency of target molecule capture across the flow cell as a whole.

FIG. 2 is a block flow diagram depicting a method for method for sequencing a target molecule in the presence of mispids, in accordance with certain example embodiments.

FIG. 3A is a graph showing a size exclusion chromatography elution profile of a 1:200 MSP2N2:DPhPE mispid preparation, in accordance with certain example embodiments. As shown, the main peak of the mispid preparation roughly corresponds to elution at the void volume, indicating the very large and inhomogeneous distribution of the particle.

FIG. 3B is an image showing gel electrophoresis of the fractions eluted from size exclusion chromatography purification of 1:200 MSP2N2:DPhPE mispid preparation (from FIG. 3A), in accordance with certain example embodiments. All lanes contain the MSP2N2 protein at approximately 40 kDa.

FIGS. 4A-4C are a series of histograms showing sequencing metrics for Xpandomer (“XP”) molecules in the presence of three concentrations of mispids as compared to Xpandomer sequencing alone, in accordance with certain example embodiments. More particularly, FIG. 4A shows throughput for three concentrations of mispid/Xpandomer conditions and the Xpandomer control. FIG. 4B shows arrival rates for the three concentrations of mispid/Xpandomer conditions and the Xpandomer control. FIG. 4C shows the accuracy for the three concentrations of mispid/Xpandomer conditions and the Xpandomer control.

FIGS. 5A-5C are a series of heatmaps showing the spatial distribution of different metrics describing Xpandomer capture along the entirety of a flow cell, in accordance with certain example embodiments.

FIGS. 6A-6C include a heatmap (FIG. 6A) showing spatial distribution of Xpandomer molecule capture frequency along the flow cell and histograms (FIGS. 6B-6C) for Xpandomer capture metrics in the presence of increasing concentrations of saposin (lipid binding protein only), in accordance with certain example embodiments.

FIGS. 7A-7B include a heatmap (FIG. 7A) showing spatial capture distribution of an alternate molecule, the fauXmer, in the presence of increasing mispid concentration and histograms (FIG. 7B), showing the overall number of fauxmer (mt_count), in accordance with certain example embodiments.

FIGS. 8A-8B include histograms (FIG. 8A) showing the overall number of high quality Xpandomer captures and a heatmap (FIG. 8B) showing Xpandomer capture across the flow cell, in accordance with certain example embodiments.

### DETAILED DESCRIPTION OF THE INVENTION

### Overview

As described herein, lipid binding molecules and/or mispids including the lipid binding molecules are used modify the interaction of a target molecule with a lipid membrane. The target molecule can be a nucleic acid sequence, for example, for which use of the lipid binding molecules and/or mispids improves sequencing efficiency and throughput of nanopore-based sequencing systems. To sequence a target molecule, such as a nucleic acid sequence, lipid binding molecules and/or mispids thereof are combined with the target molecule. The mixture is then applied to a nanopore-based sequencing chip. The target molecule is then sequenced in the presence of the lipid binding molecules and/or mispids, thereby improving the capture, arrival time, and effective concentration of the target molecule across the membrane of the chip. Such improved efficiency is particularly beneficial, for example, when concentrations of a target molecule are low.

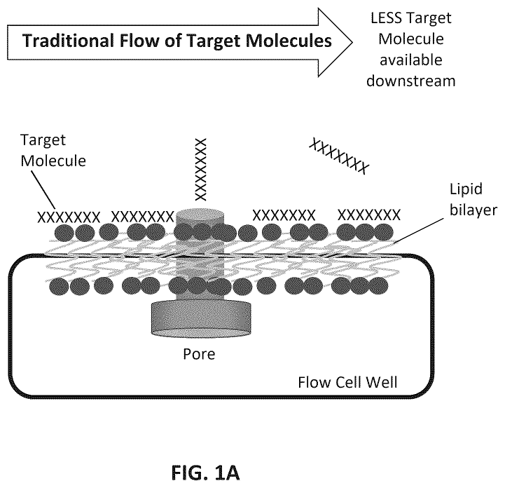

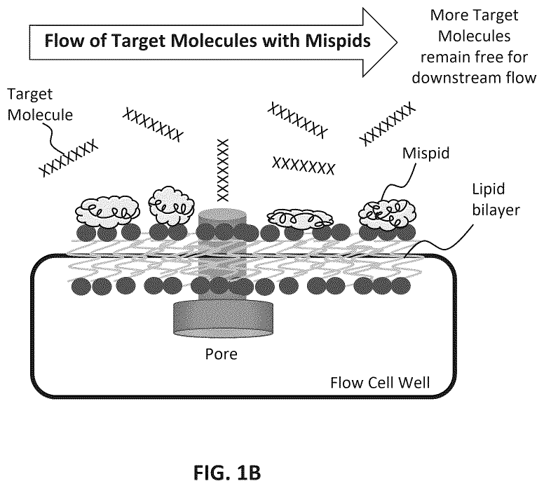

More particularly, the various embodiments described herein can be used with any biological system in which a target molecule, such as a target molecule with affinity to lipid membrane, is delivered to a nanopore via an interaction of the target molecule with the lipid membrane. This includes traditional nanopore-based sequencing chips, in which a target molecule (e.g., DNA to be sequenced) flows over a lipid-based membrane. For example, and without wishing to be bound by any particular theory, as target molecules are applied to a flow cell, those having affinity to lipids have been found to accumulate on the lipid membrane at the wells nearest to the flow cell inlet-likely because of the affinity of the target molecules to the lipid bilayer of the membrane. This is illustrated in FIG. 1A, where the target molecules are shown interacting with the lipid membrane of a flow cell well. This in turn reduces the number of target molecules available to flow across the cell (FIG. 1A). And, as more and more of the target molecules accumulate, the free flow of the target molecules across the membrane is hindered, thereby decreasing the uniform migration and distribution of the target molecules across the membrane. Consequently, the distribution of nanopore-captured target molecules is less uniform across the membrane, resulting in less efficient sequencing and reduced throughput (FIG. 1A).

To address these and other problems and inefficiencies associated with nanopore-based sequencing methods, as described herein the target molecule is combined with a solution including mispids, i.e., a polydisperse mixture of lipid-based complexes, each complex including one or more lipids/lipid bilayers and one or more lipid binding proteins. The target molecule/mispid mixture is then applied to the inlet of nanopore-based sequencing chip, for example, so that it flows across the chip with the target molecules. In other examples, the target molecule is mixed with a solution of the lipid binding proteins alone, for example, and then flowed over the lipid membrane of a nanopore based sequencing chip. In other examples, the target molecule is mixed with a nanodisc solution, i.e., a generally monodisperse and stable mixture of lipid bilayers contained within a lipid binding protein.

Without wishing to be bound by any particular theory, it is believed that the lipid binding molecules and/or the mispids bind to the lipid membrane of the nanopore-based chip, thereby preventing the target molecules from prematurely binding to the lipid membrane at the wells nearest the flow cell channel inlet. This is shown in FIG. 1B in which the mispids, for example, are depicted as binding to the lipid membrane, thus competing with the target molecules for binding to the lipid membrane of the flow cell well. The in turn increases the number of target molecules that are free to flow across the flow cell as a whole (FIG. 1B). In some examples, it is further believed that the target molecules interact with the lipid binding molecules and/or a mispids, for example, thereby further reducing undesirable interactions between the target molecule and the lipid bilayer of the chip.

Regardless, use of the lipid binding molecules and/or a mispids as described herein permits a more uniform distribution of the target molecules across the membrane (and hence a more uniform target molecule capture by a greater number of nanopores). Indeed, by applying the lipid binding proteins and/or mispids to the membrane, improved flow of target molecules over the membrane of the nanopore-based sequencing chip is achieved. This includes improved the arrival rate of target molecules to the nanopores and improved capture uniformity. Further, when concentrations of the target molecules are low, use of the lipid binding proteins and/or mispids described herein can additionally improve the evenness of flow of the limited target molecules, thereby increasing the otherwise typically decreased sequencing rate of the low-concentration sample.

Additionally or alternatively, in certain examples the lipid binding molecules and/or mispids can be applied to the nanopore-based chip before application of the target molecule. In such examples, it is believed that the lipid binding molecules and/or the mispids bind to the lipid membrane of the nanopore-based chip, thereby preventing the target molecules from prematurely binding to the lipid membrane at the well or channel inlet as described above. Thereafter, fewer of the target molecules-when applied to the chip-interact with the lipid bilayer of the chip. This similarly provides the improvements noted above, i.e., a more uniform distribution of the target molecules across the membrane (and hence a more uniform target molecule capture by a greater number of nanopores).

In certain examples, the target molecule is a nucleic acid, such as a DNA molecule to be sequenced. In certain examples, the target molecule is a modified nucleic acid sequence, such as a modified DNA sequence. In certain examples, the target molecule is surrogate nucleic acid polymer, the sequencing of which determines the sequence of an underlying target nucleic acid sequence of interest. For example, the surrogate nucleic acid polymer can rely on sequencing by expansion (SBX) technology, which includes the use of Xpandomers™ to create the surrogate nucleic acid polymer. But while Xpandomer (“XP”) sequences are ideally suited for nanopore-based sequencing, the size of these large molecules and/or other modifications to the Xpandomer sequence is believed to increase their affinity to the lipid bilayer of the well of the chip. Hence, the lipid binding peptides and/or mispids as described herein are especially useful in improving Xpandomer (SBX) based sequencing.

In certain examples, the lipid binding molecule is a lipid binding protein, such as a membrane scaffolding protein (MSP) that has affinity to the lipid membrane. For example, the scaffolding protein can be an apolipoprotein or a derivative or variant thereof (such as apolipoprotein A-I (Apo-AI) or a variant of derivative thereof). Such apolipoproteins and other nanodisc forming peptides are amphipathic molecules that, for example, form a band around a lipid bilayer to form a nanodisc. That is, the same MSPs used to make nanodiscs can be used to make the mispids described herein.

In certain examples, the amino acid sequence and/or length of the MSP can be varied to alter the lipid binding capacity of the MSP, with shorter proteins generally having less affinity for lipids (and hence forming smaller lipid nanoparticles). For examples, the MSP sequence may be adjusted such that the MSP includes a predetermined secondary structure that optimizes interactions with the lipid component of the mispids. Other parameters can also be adjusted to alter the lipid binding capacity of the MSP, such as the pH and/or salt concentration of any MSP-lipid containing solution and/or the flow rate. In certain examples, the lipid binding capacity of the MSP is adjusted so that it forms mispids but without binding lipids so strongly that it disrupts the lipid bilayer membrane of the chip.

The lipids used for preparation of the mispids described herein can be any lipids used generally for the preparation for the preparation of mispids. For example, the lipids can be phosphatidylcholine-based lipids, phosphoethanolamine-based lipids, or a synthetic derivatives or variants thereof. In certain examples, different lipid types can be used to form a solution of mispids including the different lipid type. The solution of different mispid types can then be mixed with the target molecules, for example, as described herein. In certain examples, the mispids can be stabilized with one or more non-lipid components, such as cholesterol. In certain examples, synthetic materials can be used instead of the lipids described herein, such as polymers (e.g., a triblock copolymer (TBC)).

In certain examples, commercially available kits for making nanodiscs, and/or components thereof, can be used to make the mispids. For example, rather than using specific ratios of MSPs to lipid necessary to generate homogenous, water-soluble lipid bilayers—such as is generally used when making nanodiscs—the amount of lipid is substantially increased for the mispids over these well-described ratios for nanodiscs. For example, the MSPs can be mixed with the lipid component at a ratio (MSP:lipid) of about 1:200 to about 1:1300 MSP to lipid, such as about 1:1200 MSP to lipid. Such ratios of MSP to lipid, for example, result in an inhomogeneous (polydispersion) mixture of mispids. This is in contrast to nanodisc mixtures, where the ratio MSP to lipid ratio is adjusted and controlled to create a monodisperse mixture of nanodiscs.

### Terms & Nomenclature

The invention will now be described in detail by way of reference only using the following definitions and examples. All patents and publications, including all sequences disclosed within such patents and publications, referred to herein are expressly incorporated by reference in their entirety.

Unless defined otherwise herein, all technical and scientific terms used herein have the same meaning as commonly understood by one of ordinary skill in the art to which this invention belongs. Such common techniques and methodologies are described, for example, in Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library, each of which provide one of skill with a general dictionary of many of the terms used in this invention. Although any methods and materials similar or equivalent to those described herein can be used in the practice or testing of the present invention, the preferred methods and materials are described. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

As used herein, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value of the range and/or to the other particular value of the range. It will be further understood that the endpoints of each of the ranges are significant both in relation to the other endpoint, and independently of the other endpoint. Similarly, when values are expressed as approximations, by use of the antecedent “about,” it will be understood that the particular value forms another aspect.

In certain example embodiments, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about. Further, terms used herein such as “example,” “exemplary,” or “exemplified,” are not meant to show preference, but rather to explain that the aspect discussed thereafter is merely one example of the aspect presented.

As used herein, a “membrane” is a component of a sensor or detection system or apparatus. As those skilled in the art will appreciate, the membrane (e.g., lipid membrane or lipid bilayer) is a thin film that separates two compartments or reservoirs (e.g., a cis chamber and a trans chamber) and prevents the free diffusion of ions and other molecules between these. In certain example embodiments, the membrane is preferably a lipid bilayer. Lipid bilayers are models of cell membranes and have been widely used for experimental purposes.

Suitable membranes are amphiphilic layers formed of amphiphilic molecules, i.e., molecules possessing both hydrophilic and lipophilic properties. Such amphiphilic molecules may be either naturally occurring, such as phospholipids, or synthetic. Exemplary amphiphilic materials include various phospholipids such as 1,2-diphytanoyl-sn-glycero-3-phosphoethanolamine (DPhPE), palmitoyl-oleoyl-phosphatidyl-choline (POPC), dioleoyl-phosphatidyl-methylester (DOPME), 1,2-diphytanoyl-sn-glycero-3-phosphatidylcholine (DPhPC)dipalmitoylphosphatidylcholine (DPPC), phosphatidylcholine, phosphatidylserine, phosphatidic acid, phosphatidylethanolamine, phosphatidylinositol, phosphatidylglycerol, and sphingomyelin. Exemplary synthetic amphiphilic molecules include such molecules as poly(n-butyl methacrylate-phosphorylcholine), poly(ester amide)-phosphorylcholine, polylactide-phosphorylcholine, polyethylene glycol-poly(caprolactone)-di-or tri-blocks, polyethylene glycol-polylactide di-or tri-blocks and polyethylene glycol-poly(lactide-glycolide) di-or tri-blocks.

As used herein, a “lipid component” is a lipid portion of a mispid that is contained within a stabilizing protein belt of a mispid.

As used herein, the term “mispid” refers to a heterogenous mixture of lipid and lipid binding protein complexes of variable size and stoichiometries, which may include the lipid available in different phases and arrangements. In certain example embodiments, the lipid binding protein includes one or more alpha helices.

As used herein, the term “nanodisc” refers to at least one phospholipid bilayer that is stabilized by at least one lipid binding molecule, such as a membrane scaffold protein. See Bayburt, T. H. et al., Reconstitution and imaging of a membrane protein in a nanometer-size phospholipid bilayer. J. Struct. Biol. (1998), 123 (1): 37-44 and Civjan, N. R. et al. Direct solubilization of heterologously expressed membrane proteins by incorporation into nanoscale lipid bilayers. BioTechniques (2003) 35:556-563, both of which are hereby expressly incorporated herein by reference in their entirety. For example, the lipid binding molecule can be a lipid binding protein, such as a membrane scaffold protein (e.g., apolipoprotein or portion thereof). Generally, nanodiscs are less than one micron in diameter. And when used in associate with sequencing reactions, the nanodiscs contain a nanopore. Further, a solution or mixture of nanodiscs refers generally to a monodisperse and stable lipid bilayers with the generally same amount of lipid per MSP belt.

As used herein, the term “nanopore protein” refers to a polypeptide subunit and multimers of subunits that can create an aperture through a membrane when an appropriate higher-order structure is formed. Nanopore protein may refer to a single polypeptide subunit of a multimeric nanopore protein or different oligomeric forms of single polypeptide subunits. A “mixture of nanopore proteins” refers to a solution that may contain a heterogenous combination of single and/or oligomeric forms of a nanopore protein. “Native nanopore protein” refers to the natural, higher-order state of subunit oligomerization that can form a functional nanopore in a membrane. Exemplary nanopore proteins, i.e., biological nanopores, include α-hemolysin, Mycobacterium smegmatis porin A (MspA), aerolysin, phi29, gramicidin A, maltoporin, OmpG, OmpF, OmpC, Vibrio cholerae cytolysin, PhoE, Tsx, and F-pilus.

A preferred nanopore protein is α-hemolysin (α-HL). α-HL is the major cytotoxic agent released by bacterium Staphylococcus aureus and the first identified member of the pore forming beta-barrel toxin family. This toxin consists mostly of beta-sheets (68%) with only about 10% alpha-helices. The h1a gene on the S. aureus chromosome encodes the 293-residue protein monomer, which forms heptameric oligomers in the cellular membrane to form a complete beta-barrel pore. The native α-HL nanopore protein is thus an assembly, i.e. oligomer, of seven α-HL protein monomers.

The term “nanopore,” as used herein, generally refers to a pore, channel, opening, or passage, of a specific size, formed or otherwise provided in a membrane, comprised of nanopore proteins. The size of the nanopore, for example, is such that when molecules of interest pass through the opening, the passage of the molecules can be detected by a change in signal, for example, electrical signal, e.g., current. The nanopore, for example, includes a nanopore protein or a group of nanopore proteins. For example, the nanopore may include seven α-HL proteins to form an α-HL nanopore.

As used herein, the term “membrane scaffold protein” refers to a protein that can stabilize a phospholipid bilayer in a mispid by binding to the bilayer periphery. In general, membrane scaffold proteins have hydrophobic faces that can associate with the nonpolar interior of a phospholipid bilayer and hydrophilic faces that favorably interact with a polar solvent such as an aqueous buffer. Membrane scaffold protein sequences may be naturally occurring, or may be engineered using recombinant techniques or constructed de novo. Naturally occurring membrane scaffold proteins include apolipoproteins, such as truncated apolipoproteins, which are components of lipoproteins. Known classes of apolipoproteins include: A (including, for example, apo A-I and apo A-II), B, C, D, E, and H. Non-naturally occurring membrane scaffold proteins include MSP1 and MSP2 described in U.S. Pat. No. 7,691,414, which is incorporated herein by reference in its entirety. An exemplary commercially available non-naturally occurring MSP is MSP1D1 available from, e.g., Sigma™. The membrane scaffold proteins can be the full-length protein, or a truncated version of the protein. Membrane scaffold protein is not intended to encompass various functional membrane proteins including, but not limited to, ion channels and other transmembrane receptors, porins, certain cell adhesion molecules, and electron transport proteins such as NADH dehydrogenase and ATP synthases.

As used herein, the term “DNA” refers to a molecule comprising at least one deoxyribonucleotide residue. A “deoxyribonucleotide,” is a nucleotide without a hydroxyl group and instead a hydrogen at the 2′ position of a β-D-deoxyribofuranose moiety. The term encompasses double stranded DNA, single stranded DNA, DNAs with both double stranded and single stranded regions, isolated DNA such as partially purified DNA, essentially pure DNA, synthetic DNA, recombinantly produced DNA, as well as altered DNA, or analog DNA, that differs from naturally occurring DNA by the addition, deletion, substitution, and/or modification of one or more nucleotides.

An “isolated” molecule is a nucleic acid molecule that is separated from at least one other molecule with which it is ordinarily associated, for example, in its natural environment. An isolated nucleic acid molecule includes a nucleic acid molecule contained in cells that ordinarily express the nucleic acid molecule, but the nucleic acid molecule is present extrachromasomally or at a chromosomal location that is different from its natural chromosomal location.

The term “nucleic acid molecule” includes RNA, DNA and cDNA molecules. It will be understood that, as a result of the degeneracy of the genetic code, a multitude of nucleotide sequences encoding a given protein such as alpha-hemolysin and/or variants thereof may be produced. The present invention contemplates every possible variant nucleotide sequence, encoding variant alpha-hemolysin, all of which are possible given the degeneracy of the genetic code.

The term “nucleotide” is used herein as recognized in the art to include natural bases (standard), and modified bases well known in the art. Such bases are generally located at the 1′ position of a nucleotide sugar moiety. Nucleotides generally comprise a base, sugar, and a phosphate group.

As used herein, the term “lipid” refers to lipid molecules that can include fats, waxes, steroids, cholesterol, fat-soluble vitamins, monoglycerides, diglycerides, triglycerides, phospholipids, sphingolipids, glycolipids, cationic or anionic lipids, derivatized lipids, and the like, as described in detail below. Lipids can form micelles, monolayers, and bilayer membranes. The lipids can self-assemble in combination with other components to form mispids. The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

The term “apolipoprotein” as used herein indicates an amphipathic protein that binds lipids to form lipoproteins. The term “amphipathic” pertains to a molecule containing both hydrophilic and hydrophobic properties. Exemplary amphipathic molecules comprise, a molecule having hydrophobic and hydrophilic regions/portions in its structure. Examples of biomolecules which are amphipathic include but not limited to phospholipids, cholesterol, glycolipids, fatty acids, bile acids, saponins, and additional lipids identifiable by a skilled person.

A “lipoprotein” as used herein indicates a biomolecule assembly that contains both proteins and lipids. More particularly, in lipoproteins, the protein component surrounds or solubilizes the lipid molecules enabling particle formation. Exemplary lipoproteins include the plasma lipoprotein particles classified under high-density (HDL) and low-density (LDL) lipoproteins, which enable fats to be carried in the blood stream, the transmembrane proteins of the mitochondrion and the chloroplast, and bacterial lipoproteins. In particular, the lipid components of lipoproteins are insoluble in water, but because of their amphipathic properties, apolipoproteins such as certain Apolipoproteins A and Apolipoproteins B and other amphipathic protein molecules can surround the lipids, creating the lipoprotein particle that is itself water-soluble, and can thus be carried through water-based circulation (e.g., blood, lymph in vivo or in vitro).

Apolipoproteins known to provide the protein components of the lipoproteins can be divided into six classes and several sub-classes, based on the different structures and functions. Exemplary apolipoprotein known to be able to form lipoproteins comprise Apolipoproteins A (apo A-I, apo A-II, apo A-IV, and apo A-V), Apolipoproteins B (apo B48 and apo B100), Apolipoproteins C (apo C-I, apo C-II, apo C-II, and apo C-IV), Apolipoproteins D, Apolipoproteins E, and Apolipoproteins H. For example, Apolipoproteins B can form low-density lipoprotein particles, and have mostly beta-sheet structure and associate with lipid droplets irreversibly, while Apolipoprotein A1 comprise alpha helices and can associate with lipid droplets reversibly forming high-density lipoprotein particles.

The term “alpha helix” or “α-helix” indicates a right-hand-coiled or spiral conformation (helix) of a polypeptide in which every backbone N—H group donates a hydrogen bond to the backbone C═O group of the amino acid four residues earlier facilitating hydrogen bonding. The alpha helix is a common secondary structure of proteins and is also sometimes called a classic Pauling-Corey-Branson alpha helix. The name 3.613-helix is also used for this type of helix, denoting the number of residues per helical turn, and 13 atoms being involved in the ring formed by the hydrogen bond. An “amphipathic helix” refers to an alpha helix characterized by a spatial segregation of hydrophobic and hydrophilic amino acid residues in hydrophobic and hydrophilic regions typically located on opposite faces of the helix which renders the alpha helix amphipathic. The clustered nonpolar residues can then stabilize and encourage lipid molecules to form lipoprotein complexes and stabilize lipid bilayer conformations underpinning the mispid construct.

As used herein, a “polymerase” refers to an enzyme that performs template-directed synthesis of polynucleotides. The term, as used herein, also refers to a domain of the polymerase that has catalytic activity. Generally, the enzyme will initiate synthesis at the 3′-end of the primer annealed to a polynucleotide template sequence, and will proceed toward the 5′ end of the template strand. A “DNA polymerase” catalyzes the polymerization of deoxynucleotides.

As used herein, the term “processivity” refers to the ability of a nucleic acid modifying enzyme to remain bound to the template or substrate and perform multiple modification reactions. Processivity is generally measured by the number of catalytic events that take place per binding event.

As used herein, “purified” means that a molecule is present in a sample at a concentration of at least 95% by weight, or at least 98% by weight of the sample in which it is contained. The term “purifying” generally refers to subjecting transgenic nucleic acid or protein containing cells to biochemical purification and/or column chromatography. The term “purified” does not require absolute purity. Rather, this term is intended as a relative term. Thus, for example, a purified or “substantially pure” protein preparation is one in which the protein referred to is more pure than the protein in its natural environment within a cell or within a production reaction chamber (as appropriate).

As used herein, “sequence identity” refers to the similarity between two nucleic acid sequences, or two amino acid sequences, and is expressed in terms of the similarity between the sequences, otherwise referred to as sequence identity. Sequence identity is frequently measured in terms of percentage identity (or similarity or homology); the higher the percentage, the more similar the two sequences are. For example, 80% homology means the same thing as 80% sequence identity determined by a defined algorithm, and accordingly a homologue of a given sequence has greater than 80% sequence identity over a length of the given sequence. Example levels of sequence identity include, for example, 80, 85, 90, 95, 98% or more sequence identity to a given sequence, e.g., the coding sequence for any one of the inventive polypeptides, as described herein.

Methods of alignment of sequences for comparison are well known in the art. Various programs and alignment algorithms are described in: Smith & Waterman Adv. Appl. Math. 2:482, 1981; Needleman & Wunsch J. Mol. Biol. 48:443, 1970; Pearson & Lipman Proc. Natl. Acad. Sci. USA 85:2444, 1988; Higgins & Sharp Gene 73:237-244, 1988; Higgins & Sharp CABIOS 5:151-153, 1989; Corpet et al. Nuc. Acids Res. 16, 10881-90, 1988; Huang et al. Computer Appls. In the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, MD) and on the Internet, for use in connection with the sequence analysis programs that include, for example, the suite of BLAST programs, such as BLASTN, BLASTX, and TBLASTX, BLASTP and TBLASTN.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997).

In certain example embodiments, a preferred alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

### Methods, Systems, and Compositions

Provided herein are methods, systems, and compositions for improving sequencing efficiency and throughput of nanopore-based sequencing systems. As described herein, for example, such methods, systems, and compositions can be used to improve sequencing efficiency and throughput of any nanopore-based sequencing system where the target molecule may interact with and/or otherwise have an affinity to lipids, such as the lipid bilayer membrane of a nanopore-based sequencing system.

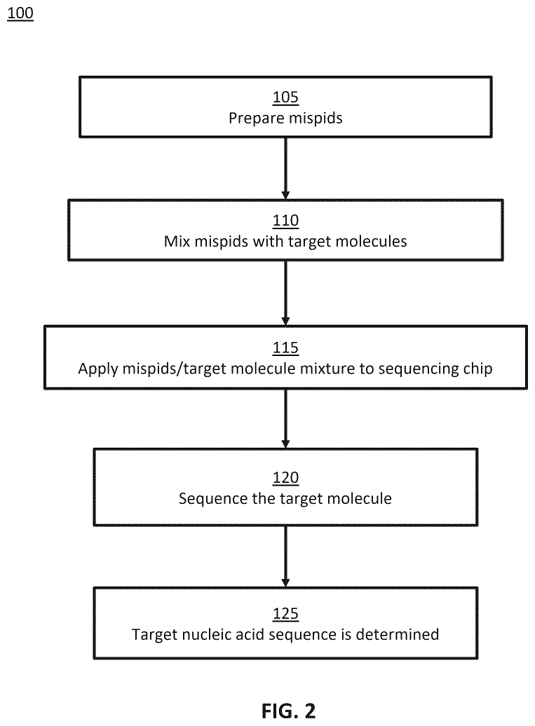

Turning to the drawings, in which like numerals indicate like but not necessarily identical elements throughout the figures, example embodiments are described in detail. More particularly, FIG. 2 is a block flow diagram depicting a method for sequencing a target molecule in the presence of mispids, in accordance with certain example embodiments. It is to be understood, however, that while FIG. 2 describes use of mispids, in certain example embodiments the lipid binding proteins described herein can be used alone or in combination with the mispids to achieve improved target molecule sequencing as described herein.

With reference to FIG. 2, in block 105, mispids compositions are prepared for use with the methods described herein. Generally, the mispids can be made according to methods known in the art for making nanodiscs, except that a higher amount of lipid is substituted for the lower amount of lipid typically used when making nanodiscs. That is, known methods for making nanodiscs can readily be used and adapted to make the mispids described herein, such as via the use of commercially available components and kits to make nanodiscs (but with larger amounts of lipids). As those skilled in the art will appreciate, preparing nanodiscs generally involves forming a vesicle of a lipid component (e.g., vesicles or detergent solubilized lipid complexes), followed by addition of a lipid binding molecule and a solubilizing detergent, typically sodium cholate. Nanodiscs are formed as the detergent is removed, either by dialysis or size exclusion chromatography, where the lipid binding molecule functions as a “belt,” for example, around a lipid bilayer of the lipid components, thereby stabilizing the overall nanodisc structure. These same techniques can be used to make the mispids described herein, with modifications such that mixture is a polydisperse mixture of lipid-based complexes, each complex including one or more lipids and one or more lipid binding proteins.

For example, the lipid binding molecule can be any naturally occurring or synthetic molecule (e.g., DNA, peptide, or other compound) that, for example, assumes an alpha helical structure and interacts or inserts with a lipid bilayer. When the lipid binding molecule is a protein, for example, the protein can be synthesized by methods known in the art. When the lipid binding molecule is DNA, for example, such molecules can likewise be prepared according to methods known in the art, including for example as described in Zhao et al., J. Am. Chem. Soc. 2018, 140, 34, 10639-10643, which is hereby incorporated herein by reference in its entirety.

In certain example embodiments, the lipid binding molecules are membrane scaffolding proteins (MSP), such as apolipoprotein or a derivative or variant thereof (e.g., apolipoprotein A-I (Apo-AI) or a variant of derivative thereof). Example MSPs include, for example, Membrane Scaffold Protein 1D1 (MSP1D1), Membrane Scaffold Protein 2N2 (MSP2N2), Membrane Scaffold Protein 1E3D1 (MSP1E3), each of which is commercially available from Sigma-Aldrich™. Additionally or alternatively, the lipid biding molecules may include derivatives of saposin A, amyloid beta peptides, alpha-synuclein derivatives, styrene malic anhydride (SMA) acid derivatives that solubilize lipid bilayers, diisobutylene-malic acid (DIBMA) derivatives, and Poly acrylic acid-co-styrene derivatives. In certain example embodiments, the lipid binding molecule is a synthetic apolipoprotein, i.e., a non-naturally occurring apolipoprotein, such as any of those set forth in U.S. Pat. No. 11,279,749, which is hereby incorporated herein by reference in its entirety.

In certain example embodiments, the lipid binding molecules include proteins having a predetermined number of alpha helices. As those skilled in the art will appreciate, such alpha helices include a right-hand-coiled or spiral conformation (helix) of a polypeptide in which every backbone N—H group donates a hydrogen bond to the backbone C═O group of the amino acid four residues earlier facilitating hydrogen bonding. The alpha helix is a common secondary structure of proteins and is also sometimes called a classic Pauling-Corey-Branson alpha helix. The name 3.613-helix is also used for this type of helix, denoting the number of residues per helical turn, and 13 atoms being involved in the ring formed by the hydrogen bond.

In certain example embodiments, the lipid binding proteins include 3 to 10 alpha helices, such as 3, 4, 5, 6, 7, 8, 9 or 10 alpha helices. For example, shorter proteins, such as shorter MSP1D1 proteins, can be made by deleting or partially deleting one or more of the MSP1D1 alpha helices, such as is described in Hagn et al., Nat Protoc. 2018 January; 13 (1): 79-98, which is hereby incorporated herein in its entirety. That is, the number of alpha helices may be modified to determine the size of the mispid. For example, and with reference to MSP1D1, the proteins may include a ΔH4/2 deletion, a ΔH4 deletion, a ΔH5 deletion, a ΔH4/2ΔH5 deletion, a ΔH4H5 deletion, and/or a ΔH4-H6 deletion. See Hagn et al., Nat Protoc. 2018 January; 13 (1): 79-98. The different sized mispids made from such modified MSP1D1 proteins, for example, have an altered lipid binding capacity to a lipid bilayer

Without wishing to be bound by any particular theory, it is believed that smaller lipid binding proteins and mispids made therefrom will have lower affinities for the nanopore bilayer than those made with larger lipid binding proteins. Hence, in certain example embodiments, adjusting the size of the lipid binding protein and/or mispids can be used, for example, to tune and modulate target molecule capture and distribution during flow over a membrane, such as over the membrane of a flow cell. In other words, using lipid binding proteins to control and manage the size of the mispids made therefrom, for example, can ultimately be used to adjust the flow rate of a membrane of a well, thereby tailoring (or tuning) the flow rate of the mispids—and hence target molecules—to a given application.

In certain example embodiments, the (i) length of the lipid binding proteins, and/or (ii) the size of the mispids made therefrom, and/or (iii) the flow rate at which the lipid binding proteins and/or mispids are delivered, can each be adjusted to determine the flow rate of target molecule over a lipid membrane. That is, each of these parameters can be adjusted to modulate the distribution of target molecule capture over the surface of the entire flow cell surface. In other words, by manipulating both the length of the lipid binding proteins/mispids- and the flow rate at which they are delivered to the bilayer with the target molecule-sequencing efficiency across the entire flow cell can be tuned with respect to the target molecules affinity for the bilayer and to improve capture across the flow cell.

Methods of preparing nanodiscs, which can be readily modified and adapted as described herein to form mispids, are described generally in Li et al., Preparation of Lipid Nanodiscs with Lipid Mixtures, Curr Protoc Protein Sci. 2019 December; 98 (1): e100, which is hereby expressly incorporated herein in its entirety; see also Hagn et al., Nat Protoc. 2018 January; 13 (1): 79-98. In certain example embodiments, mispids are prepared by mixing a lipid binding protein (MSP) with a lipid of choice in specific ratios in the presence of a solubilizing detergent (sodium cholate) followed by detergent removal, either by dialysis or the use of biobeads. Additionally or alternatively, preparing mispids can involve a cell-free method, such as those methods described in U.S. Pat. No. 11,053,322 (for nanodiscs), which is hereby incorporated herein by reference in its entirety.

In certain example embodiments, components to make nanodiscs—and hence the components to make mispids—can be purchased from Merck KGaA™ Cube Biotech™, Anatrace Products, LLC™, Mempro™, and Avanti Lipids™. Additionally or alternatively, customizable kits for nanodisc assembly are generally available, such as those from Cube Biotech™ (which uses the membrane scaffolding protein MSP2N2 and the phospholipid DMPC as the primary component of the lipid-bilayer of the mispid) and Mempro™. With the Mempro™ kit, for example, self-assembly is used to in a two strep process, whereby phospholipids are first dissolved by a detergent into mixed lipid-detergent micelles. The mixed micelles are then transformed into phospholipid bilayers by incubating with an MSP and thereafter removing the detergent using beads. Removal of the detergents alters the effective packing parameter of the detergent-lipid micelles from spherical to a planar bilayer geometry, with the planer bilayer being stabilized by the addition of the MSPs.

While traditional nanodisc formation relies on optimal molar ratios of lipid binding molecules (e.g., MSPs) to lipids to form stable lipid bilayers and accounts for the type of lipid, the lipid's phase behavior, and the lipid's phase temperature, the mispids described herein by increasing the amount of lipid relative to the amount of the lipid binding molecules to form a variety of lipid-MSP complexes that may or may not include stable lipid bilayers. Accordingly, in certain example embodiments mispids for use with the methods and systems described herein have a ratio of lipid biding molecule (e.g., MSPs) to lipid of approximately 1:500, 1:600, 1:700, 1:800, 1:900. 1:1000; 1:1100, 1:1200, 1:1300, 1:1400, 1:1500, 1:1600, 1:1700, 1:1800, 1:1900, 1:2000, 1:2100, or 1:2200. This is in contrast to nanodiscs, where the ratio of lipid binding protein to lipids is 1:100, such as 1:50, 1:60, 1:70, 1:80, or 1:90.

In certain example embodiments, the molar ratio of lipid binding molecule (e.g., MSPs) to lipid is 1:1000; 1:1100, 1:1200, 1:1300, 1:1400, such as about 1:1200. In certain example embodiments, the mispids are an inhomogeneous mixture of lipid binding molecule to lipid component ratios (i.e., lipid binding molecule:lipid component), such as a mixture of those with a ratio from about 1:500 to about 1:2200 as described hereon, or from about 1:1000 to 1:1400, such as a mixture with ratios of 1:1100, 1:1200, 1:1300, and/or 1:1400. In other words, the mispids for use in the methods described herein can include those with a mixture of different lipid binding component to lipid ratios.

In certain example embodiments, the lipid binding molecule is an MSP2N2 protein while the lipid of DPhPE. In such example embodiments, the molar ratio of MSP2N2:DPhPE is 1:200, 1:500, 1:600, 1:700, 1:800, 1:900. 1:1000; 1:1100, 1:1200, 1:1300, 1:1400, 1:1500, 1:1600, 1:1700, 1:1800, 1:1900, 1:2000, 1:2100, or 1:2200. In certain example embodiments, the molar ratio of MSP2N2:DPhPE is 1:1000; 1:1100, 1:1200, 1:1300, 1:1400, such as about 1:1200. In certain example embodiments, the mispids are a inhomogeneous mixture of MSP2N2:DPhPE ratios, such as a mixture of those with a ratio from about 1:500 to about 1:1700 or from about 1:1000 to 1:1400, such as a mixture with ratios of 1:1100, 1:1200, 1:1300, and/or 1:1400. Of course, other MSP proteins and/or variants thereof can be used with these ratios, such as MSP1D1, MSP1E3D1, and/or MSP1D1ΔH5, with the lipid component being DPhPE, POPC, DOPME, DPhPC, and/or DPPC, as described herein.

In certain example embodiments, the size of the mispid can be manipulated by adjusting the ratio of lipid binding protein to lipid component. Without being bound by any particular theory, it is believed that higher ratios of lipid binding protein to lipid component (e.g., decreased amount of lipid vs. the amount of lipid binding protein) result in smaller mispids that have a decreased affinity to a given lipid bilayer. This in turn allows greater flow of the smaller mispids across the membrane, which can in turn more evenly (and rapidly) distribute the mispids across the membrane, thereby increasing target molecule capture. Conversely, by increasing the amount of lipid component (i.e., by decreasing the ratio of lipid binding protein to lipid), it is believed that the resultant larger mispids have an increased affinity to a given lipid bilayer and hence move more slowly across the flow cell, while nonetheless having more interaction with the membrane and hence freeing more target molecules to move across the cell. Hence, depending on the application, the nature of the target molecule, the desired flow rate, and the desired target molecule nanopore capture rate, the ratios of the lipid binding protein to the lipid component can be adjusted. In certain example embodiments, for example, a mixture of mispids with different ratios of lipid binding protein to lipid component may be preferable. In certain example embodiments, nanodiscs can be substituted for the mispids to as described herein.

In block 110, the mispids are mixed with the target molecule. That is, the mispids are combined with the target molecule, such as before the target molecule is to be added to a nanopore based sequencing chip. For example, the mispids described herein can be mixed with the target molecule in any suitable buffer. Non-limiting examples of buffers include phosphates (e.g., PBS), citrates, acetates, glutamates, carbonates, tartrates, triethanolamine (TRIS), glycylglycine, histidine, glycine, lysine, arginine, and other organic acids. More specifically, non-limiting examples of buffers include Sodium HEPES, MES, Potassium Phosphate, Potassium Thiocyanate, Sterilant, TAE, TBE, Ammonium Sulfate/HEPES, BuffAR, Sodium Acetate, Sodium Carbonate, Citric Acid Sodium, Sodium Dihydrogen Phosphate, Disodium Hydrogen Phosphate, and Sodium Phosphate. In certain example embodiments, the buffer is 20 mM Tris HCl (pH 8) and 200 mM NaCl; 100 mM PBS (pH 7.4); 50 mM MES (pH 5.5) 200 mM NaCl; and the like.

Additionally or alternatively, in certain example embodiments the mispids and target molecules may be combined in the flow cell inlet of the sequencing chip, without any mixing of the target molecules and the mispids before application to the sequencing chip. That is, the mispids and target molecules can be separately added to the flow-cell inlet of the sequencing chip, allowing the mispids and target molecules to mix with each other in the flow cell inlet. In such example embodiments, a flow cell buffer can be used to suspend the target molecules and the mispids. Additionally or alternatively, the mispids may first be applied to the flow cell inlet and allowed to flow across the flow cell, followed by application of the target molecules to the flow-cell inlet (for flowing across the flow cell). That is, the mispids and the target molecules can be added sequentially.

The target molecule can be any nucleic acid sequence, modified nucleic acid sequence, and/or a nucleic acid surrogate, for example, for which detecting the molecule and/or determining the target molecule sequence is desired. For example, the target molecule may be a DNA sequence or RNA sequence, or in some instances a DNA sequence or RNA sequence that has been modified for improved sequencing or other applications. For example, U.S. Pat. No. 10,851,405 describes a modified target molecule that includes a hydrophobic capture element. The hydrophobic capture element, for example, increases the affinity of the target molecule to the lipid membrane and hence improves nanopore capture of the target molecule. See 10,851,405, which is hereby incorporated herein in its entirety. Hence, the target molecule, as used herein, can be a modified target molecule, such as a target molecule including a hydrophobic capture element or other feature that modulates affinity of the target molecule to the membrane. In other example embodiments, the target molecule may include a label, tag, or other molecule attached thereto, for example, that assists with and/or aids in sequencing.

In certain example embodiments, the target molecule can be a nucleic acid surrogate, the decoded sequence of which detects and/or is indicative of a target nucleic acid sequence of interest. That is, when the sequence of the nucleic acid surrogate is determined—by decoding the sequence of the surrogate—the determined sequence identifies and provides a nucleic acid sequence corresponding to a target nucleic acid sequence of interest. In other words, the nucleic acid surrogate serves as a substitute sequence, for example, for the target sequence of interest.

In certain example embodiments, the nucleic acid surrogate is based on Xpandomer technology, which relies on a nanopore-based Sequencing-by-Expansion (Nano-SBX) for decoding the sequence of the Xpandomer. See, e.g., U.S. Pat. No. 7,939,259, titled “High Throughput Nucleic Acid Sequencing by Expansion;” and PCT publication WO2020236526A1, titled “Translocation control elements, reporter codes, and further means for translocation control for use in nanopore sequencing,” both of which are hereby incorporated herein in their entirety. In general terms, SBX uses biochemical polymerization to transcribe the sequence of a DNA template onto a measurable polymer called an “Xpandomer.”

More particularly, the SBX technique is based on the polymerization of highly modified, non-natural nucleotide analogs referred to as “XNTPs.” The transcribed sequence, with the XNTPs, is encoded along the Xpandomer backbone in high signal-to-noise reporters that are separated by ~10 nm and are designed for high-signal-to-noise, well-differentiated responses. These differences provide significant performance enhancements in sequence read efficiency and accuracy of Xpandomers relative to natural DNA. The XNTPs, for example, are expandable, 5′ triphosphate modified non-natural nucleotide analogs compatible with template dependent enzymatic polymerization. Each XNTP has two distinct functional regions, i.e., a selectively cleavable phosphoramidate bond linking the 5′ α-phosphate to the nucleobase and a symmetrically synthesized reporter tether (SSRT) that is attached within the nucleoside triphosphoramidate at positions that allow for controlled expansion by cleavage of the phosphoramidate bond. The SSRT includes linkers separated by the selectively cleavable phosphoramidate bond. Each linker attaches to one end of a reporter code. While the XNTP substrates are bound to the daughter strand via template-dependent polymerization, they are present in a “constrained configuration” due to their size. The constrained configuration of polymerized XNTPs is the precursor to the expanded configuration, as found in Xpandomer products. The transition from the constrained configuration to the expanded configuration occurs upon scission of the P—N bond of the phosphoramidate within the primary backbone of the daughter strand.

During Xpandomer assembly, the monomeric XNTP substrates (XATP, XCTP, XGTP and XTTP) are polymerized on the extendable terminus of a nascent daughter strand by a process of template-directed polymerization using single-stranded template as a guide. This process is initiated, for example, from a primer and it proceeds in the 5′ to 3′ direction. Generally, a DNA polymerase or other polymerase is used to form the daughter strand, and conditions are selected so that a complimentary copy of the template strand is obtained. After the daughter strand is synthesized, the coupled SSRTs form the constrained Xpandomer that further forms the daughter strand. SSRTs in the daughter strand have the “constrained configuration” of the XNTP substrates. The constrained configuration of the SSRT is the precursor to the expanded configuration, as found the Xpandomer product.

The transition from the constrained configuration to the expanded configuration results from cleavage of the selectively cleavable phosphoramidate bonds (illustrated for simplicity by the unshaded ovals) within the primary backbone of the daughter strand. In this embodiment, the SSRTs include one or more reporters or reporter codes, i.e., A, C, G, or T, specific for the nucleobase to which they are linked, thereby encoding the sequence information of the template (as a surrogate). In this manner, the SSRTs provide a means to expand the length of the Xpandomer and lower the linear density of the sequence information of the parent strand.

In certain example embodiments, the target molecule can be a nucleic-acid based reporter region that facilitates identification of a target molecule. That is, the target molecule can be a portion of a lager sequence, the sequencing of which detects or otherwise identifies the presence of the target molecule. For example, the target molecule may include, as a reporter region, an address region and a probe region. See, e.g., U.S. Pat. No. 9,850,534, which is incorporated herein in its entirety. In such example embodiments, sequencing the address region while the target molecule is directed through the nanopore to determine a nucleic acid sequence of the address region identifies the target molecule based upon a nucleic acid sequence of the address region.

In block 115, a nanopore-based sequencing chip is contacted with the mixture of mispids and target molecules. That is, the mixture of the mispids and target molecule to be sequenced are applied to the flow-cell inlet of the chip (or other port or region of the chip where the sample is to be deposited), thereby contacting the chip with the mixture. For example, when the mispids and target molecules are mixed in a solution, a portion of the solution is applied to the flow-cell inlet of the chip, thereby allowing the mispids and target molecules to flow across the chip in, for example, a sequencing reaction.

Further, because the mispids as described herein improve the capture, arrival time, and effective concentration of the target molecule in nanopore-based sequencing chips that include a lipid membrane, it is contemplated that any chip having such a lipid membrane can be used with the methods and systems described here. That is, the methods and systems described herein can be beneficially used with hydrophobic bilayer, particularly a lipid bilayer. This includes, for example, example chips with the CMOS manufactured by TSMC and the microwell manufactured by TSI (in Roseville).

As those skilled in the at will appreciate, nanopore-based chip devices for detecting nucleic acids have been developed for rapid sequencing and various designs and methods of use are known in the art. See, e.g., U.S. Pat. No. 9,494,554B2, U.S. Pat. No. 9,567,630B2, U.S. Pat. No. 9,557,294B2, U.S. Pat. No. 9,605,309B2, each of which is hereby incorporated by reference herein in their entirety. These devices generally comprise an electrochemical cell with a chamber containing a nanopore embedded in a membrane. The membrane acts to separate the cell chamber into two sub-chambers, referred to as the cis and trans sides of the cell, each of which contain an electrode. The membrane can be an organic membrane, such as a lipid bilayer, or a synthetic membrane made of a non-naturally occurring polymeric material.

The pore of the nanopore acts as a channel (or passage) in the membrane between the cis and trans sides of the cell. Depending on the nanopore used, the pore has a width or diameter that can range from about 1 angstrom to about 10,000 angstroms. The nanopore can be a naturally-occurring pore-forming protein, such as α-hemolysin from S. aureus, non-naturally occurring mutant, or variant of a wild-type pore-forming protein. A range of naturally and non-naturally occurring nanopores having varying pore-sizes and properties are known in the art. See, e.g., U.S. Pat. No. 10,351,908B2, U.S. Pat. No. 10,934,582B2, U.S. Pat. No. 10,227,645B2, each of which is hereby incorporated herein in their entirety. Within the electrochemical cell, the nanopore embedded in the membrane is disposed in proximity to an electrode coupled to a sensing circuit, such as, for example, a complementary metal-oxide semiconductor (CMOS) or field effect transistor (FET) circuit. Sensing electrodes determine the signals arising from the nanopore as a target molecule, for example, traverses the nanopore from the cis to the trans side.

Electrochemical cells for nanopore-based sequencing of nucleic acids are typically used in a massively parallel fashion in which thousands of such cells are configured as an array in a single device often referred to as a chip (or bio-chip). Indeed, it is contemplated that the use of mispid as described herein to improve sequencing efficiency and throughput can be used with nanopore-based sequencing by synthesis (Nano-SBS). A typical nanopore-based sequencing chip device incorporates an array of one million or more electrochemical cells, and it may include 1000 rows by 1000 columns of such cells (See e.g., chips fabricated by Roche Sequencing Solutions, Santa Clara, CA, USA). Methods for fabricating and using such nanopore array microchips (Nano-SBS) can also be found in U.S. Patent App. Pub. Nos. 2013/0244340 A1, US 2013/0264207 A1, US2014/0134616 A1, 2015/0368710 A1, and 2018/0057870 A1, 20190085386A1, and published International Application WO 2019/166457 A1, each of which is hereby incorporated by reference herein in its entirety.

Each well in the array can be manufactured using a standard CMOS process with surface modifications that allow for constant contact with biological reagents and conductive salts. Each well can support a phospholipid bilayer membrane with a nanopore conjugate embedded therein, as part of an integrated circuit. The integrated circuit may be an application specific integrated circuit (ASIC). In certain example embodiments, the integrated circuit is a field effect transistor or a CMOS device. The sensing circuit may be situated in a chip or other device having the nanopore, or off of the chip or device, such as in an off-chip configuration. The semiconductor can be any semiconductor, including, without limitation, Group IV (e.g., silicon) and Group III-V semiconductors (e.g., gallium arsenide). See, e.g., WO 2013/123450. Further, the electrode at each well is individually addressable by computer interface. All reagents used are generally introduced into a simple flow cell above the array microchip using a computer-controlled syringe pump. The chip supports analog to digital conversion and reports electrical measurements from all electrodes independently at a rate of over 1000 points per second. Nanopore measurements can be made asynchronously at each of 8 M addressable nanopore-containing membranes in the array at least once every millisecond (msec) and recorded on the interfaced computer.

In block 120, the target molecule is sequenced. That is, a sequencing reaction is carried out on the chip, such as via the chip manufacturer's protocol or other protocols known in the art. For example, as a solution including the mixture of mispids and target molecule is applied to the chip, the solution-including the mixture of mispids and target molecule therein-moves or “flows” across the chip, bringing the target molecules and mispids into contact with the lipid membranes of the wells of the chips. Thereafter, when a voltage potential is applied (via the electrodes) across a nanopore immersed in a conducting fluid, a small current attributed to the flow of ions through the nanopore can be observed. This ion flow is sensitive to the pore size, and thus, molecules entering the pore affect the ion flow and the voltage measured through this sensor circuit. When the target molecule is an Xpandomer, for example, as the Xpandomer product elongates and passes through the nanopore, excited fluorophores associated with the Xpandomer emit detectable signals. The signals are temporally spaced as a function of the length of the tether and the speed of the Xpandomer passing through the nanopore. See, e.g., U.S. Pat. No. 7,939,259.

In block 125, the target nucleic acid sequence is determined. That is, the nucleotide sequence of the underlying target nucleic acid is determined (e.g., decoded) from the signals associated with the sequencing reaction, such as by using techniques known in the art. See, e.g., U.S. Pat. No. 7,939,259, U.S. Patent App. Pub. Nos. 2013/0244340 A1, US 2013/0264207 A1, US2014/0134616 A1, 2015/0368710 A1, and 2018/0057870 A1, 20190085386A1, and published International Application WO 2019/166457 A1. Further, if the target molecule is a DNA strand and the non-coding strand is sequenced, the sequence of the coding strand can be determined, thereby providing the nucleic acid sequence of the target nucleic acid sequence. Likewise, if the target molecule is an Xpandomer, the sequence of the Xpandomer is used to determine the underlying nucleic acid sequence.

While the above example embodiments pertain to the preparation and use of mispids for use with the methods provided herein, in certain example embodiments the lipid binding molecules can be mixed with the target molecules to improve sequencing efficiency and throughput of nanopore-based sequencing systems, without preparing the mispids as described in block 105. That is, in certain example embodiments the lipid binding molecules described herein are used alone with the target molecules, without the need for using the mispids. In such example embodiments, the lipid binding molecule can be any of the lipid binding molecules described herein, such as, for example, any of the MSPs described herein.

Further, in such example embodiments, the methods described herein for blocks 110-115 are varied to account for use of the lipid binding molecules. For example, in block 110, the lipid binding molecules are combined with the target molecules, such as in a buffer suitable for both the lipid binding molecules and the target molecule. Thereafter, in block 115, the mixture of lipid binding molecules and target molecules is applied to a sequencing chip. That is, the mixture is introduced into the flow cell inlet of the sequencing chip, for example, so that the mixture contacts the chip and can be flowed over the flow cell. In certain example embodiments, the lipid binding molecules and target molecules may be first mixed in the flow cell inlet, i.e., there is no mixing of the lipid binding molecules and the target molecules before they are applied to the chip (but rather they are mixed, for example, in the flow cell inlet of the chip). Regardless, once the chip is contacted with the mixture of lipid binding molecules and target molecules, the target molecule can be sequenced as set forth in block 120.

Without wishing to be bound by any particular theory, during target molecule sequencing reactions, such as when sequencing Xpandomers, it has been found that target molecules having affinity to lipids, such as the lipid bilayers of the sequencing chip, tend to accumulate at the wells nearest to the flow cell inlet. These wells are closest to the flow cell inlet, for example, and hence are the first wells exposed to such target molecules, thereby providing the initial opportunities for the target molecules to interact with the lipid bilayers thereof. As more and more of the target molecules accumulate, the free flow of the target molecules across the membrane is hindered. That is, during the sequencing reaction it is believed that the free flow of the target molecules is slowed and decreased, thereby decreasing the uniform migration and distribution of the target molecules across the membrane. Consequently, the distribution of nanopore-captured target molecules is less uniform across the membrane, resulting in less efficient sequencing and reduced throughput. And of course, in certain example embodiments both lipid binding molecules and mispids can be mixed with the target molecules, with the mixture applied to the chip and then sequenced as described herein.

By contacting the chip with the lipid binding molecules and/or mispids and conducting the sequencing reaction in the presence of the lipid binding molecules and/or mispid, the lipid binding molecules and/or mispids flow across the chip with the target molecules. And still, without being bound by any particular theory, it is believed that the lipid binding molecules and/or mispids bind to the lipid membrane of the nanopore-based chip, thereby preventing the target molecules from prematurely accumulating at the wells of the flow cell channel inlet. For example, the lipid binding molecules and/or mispids may compete with the target molecules for interacting with the well lipid bilayers, thereby reducing the interaction of the target molecules with the lipid bilayers. Additionally or alternatively, it is further believed that the target molecules interact with the lipid binding molecules and/or mispid, for example, thereby further reducing undesirable interactions between the target molecule and the lipid bilayer of the chip.

While the embodiments provided herein include those directed to nanopore-based sequencing applications, e.g., modification of the interaction between a target molecule to be sequenced and the lipid membrane of a nanopore-based sequencing chip, it is to be understood that that the methods, systems, and compositions provided herein can be used to modify interactions between a target molecule and lipid membrane generally. For example, when a target molecule is modified to include a hydrophobic capture element as described in U.S. Pat. No. 10,851,405, which is hereby incorporated herein in its entirety, the lipid binding protein and/or mispids provided herein can be used to manipulate the interaction of the modified target molecule with a lipid membrane. That is, the mispids compete with the target molecules for space on the lipid bilayer, and in the case of poor target molecule distribution across the flow cell, can be used to manipulate/improve capture distribution and reducing sequencing times. In other example embodiments, the mispids can be used to directly modify the components of the lipid bilayer, such as delivering membrane proteins and/or specific lipid components to generic lipid bilayers (lipidomics) which modify pore behavior.

As is evident from this disclosure, the methods and systems described herein are particularly useful when the concentration of the target molecules is low. For example, by sequencing a target molecule as described herein in the presence of the lipid binding proteins and/or mispids as described herein, more of the target molecule—in a given amount solution applied to the sequencing chip—is free to flow across the chip. In other words, fewer of the otherwise limited amount of target molecules bind to the lipid membrane of the wells of the chips, allowing more of the target molecules to flow down the chip to interact with a nanopore. Hence, particularly when concentrations of the target molecule are low, the methods and systems described herein can improve the evenness of flow of the limited target molecules, thereby increasing the otherwise typically decreased sequencing rate of the low-concentration sample.

These and other related embodiments will be contemplated by the skilled artisan in view of this disclosure.

### EXAMPLES

The following examples further illustrate the invention but should not be construed as in any way limiting its scope. In light of the present disclosure and the general level of skill in the art, those of skill will appreciate that the following examples are intended to be exemplary only and that numerous changes, modifications, and alterations can be employed without departing from the scope of the presently disclosed subject matter.

As used herein, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### Example 1—Preparation of Mispids

This example describes the preparation of mispids. Briefly, Membrane Scaffolding Proteins (MSPs) and MSP variants were selected to cover a broad range of lipid capacity, i.e., MSP2N2 with a high lipid binding capacity vs. MSPAH5 with a lower lipid capacity for testing.

To prepare mispids, purified MSP protein suspended in 20 mM TriHCI (pH 8)/200 mM NaCl buffer was mixed directly with lipids solubilized in sodium cholate at 1:200. Mixtures of MSPs, lipid, and sodium cholate were dialyzed overnight 20 mM TriHCI (pH 8)/200 mM NaCl buffer, below the phase transition temperature of individual lipids. Spontaneous formation of mispids occurred as detergent was removed from the mixtures.

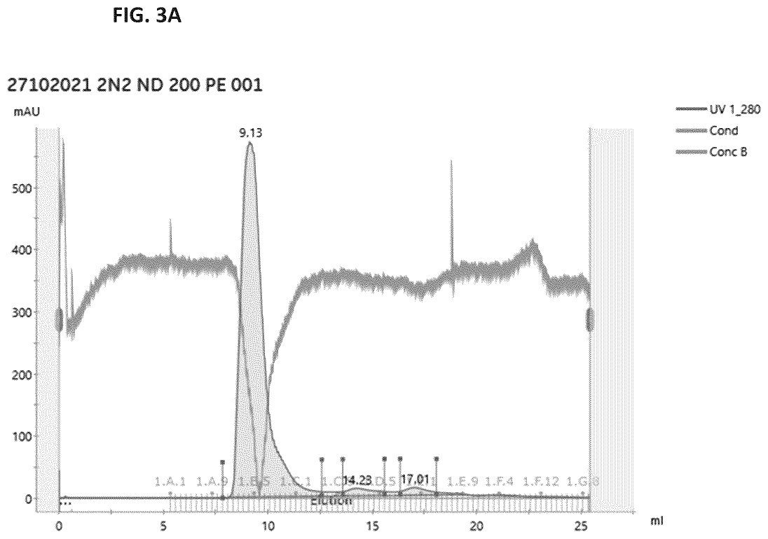

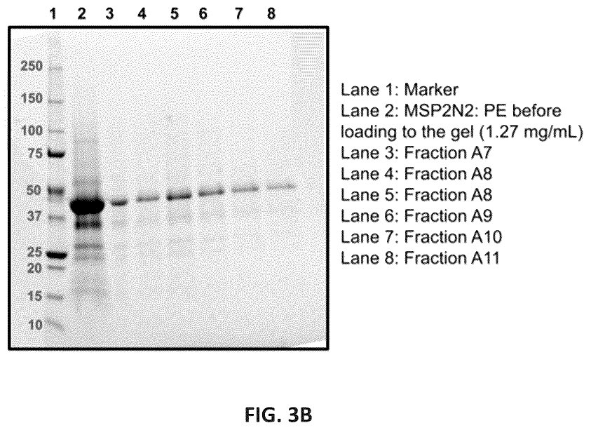

The MSP2N2:DPhPE (1:200 molar ratio) mispids were then dialyzed overnight and further purified by size exclusion chromatography using a Superdex 200 column (in 20 mM Tris pH 8 200 mM NaCl) to yield fractions of variable purity (FIG. 3A). As shown, the main peak of the mispid preparation roughly corresponds to elution at the void volume, indicating the very large and inhomogeneous distribution of the particle.

FIG. 3B shows gel electrophoresis of the fractions eluted from size exclusion chromatography purification of 1:200 MSP2N2:DPhPE mispid preparation. All lanes contain the MSP2N2 protein at approximately 40 kDa. Monodisperse and highly homogenous nanodisc fractions where the majority of the lipid and MSP are in defined stable ratios will elute from the column at well-defined retention volumes. However, less pure fractions can also be observed and used successfully in this application as demonstrated by the mispid MSP2N2 preparations. Optimal fractions are tested directly during sequencing and can be stored at 4 deg for two weeks. With large of lipid in the mixture, the formation of stable nanodiscs is greatly reduced.

### Example 2—Target Molecule Sequencing With & Without Mispids

This example describes a sequencing reaction of an Xpandomer sequence, with and without the inclusion of mispids in the sequencing reaction.

For sequencing reactions, target molecule/mispid mixture were prepared using the mispids from Example 1. Briefly, the size-exclusion purified mispids from Example 1 (20 mM TriHCl (pH 8)/200 mM NaCl) were mixed directly with Xpandomer stored in 10-40% acetonitrile/1-10% trehalose and subsequently diluted in Xpandomer dilution buffer (100 mM MES pH 6.2 842 mM Urea, 5.26% PEG8k, 0.16% trehalose, 1.05M NH4Cl, 158 mM K3Fe(CN)6, 158 mM K4Fe(CN)6). While the optimal mispid:Xpandomer ratio depends on the total lipid and MSP2N2 concentration, typical amounts used at 1-2 μL for 10 μL of Xpandomer.

For Xpandomer sequencing, sequencing was carried out according to Int'l Pat. App. WO2020236526, with mixtures of Xpandomer/mispids mixed according to Example 2 being used in the sequencing reactions including mispids. Sequencing was performed either without mispid or with varying mispid concentrations of 1.4%, 2.4% and 4.8% of the total volume of Xpandomer used and were tested. It was found that mispids improved sequencing performance without impacting sequencing accuracy. For example, a concentration of 1.4% total Xpandomer volume performed better than Xpandomer sequencing without mispid with the following median metrics: num_hqmt_count of 76 without mispid and 140 with 1.4% mispid, num_ahqmt_per_functional_min_per_cel of 2.55 without mispid and 4.72 with 1.4% mispid, extended_arrival_rate of 0.29 without mispid and 0.53 with 1.4% mispid, align_edit_pct_identical of 97.7 without mispid and 97.7 with 1.4% mispid.

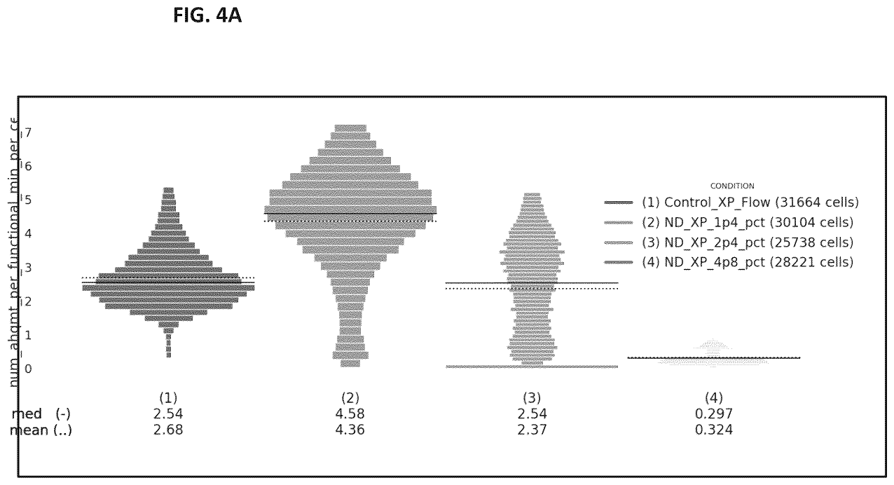

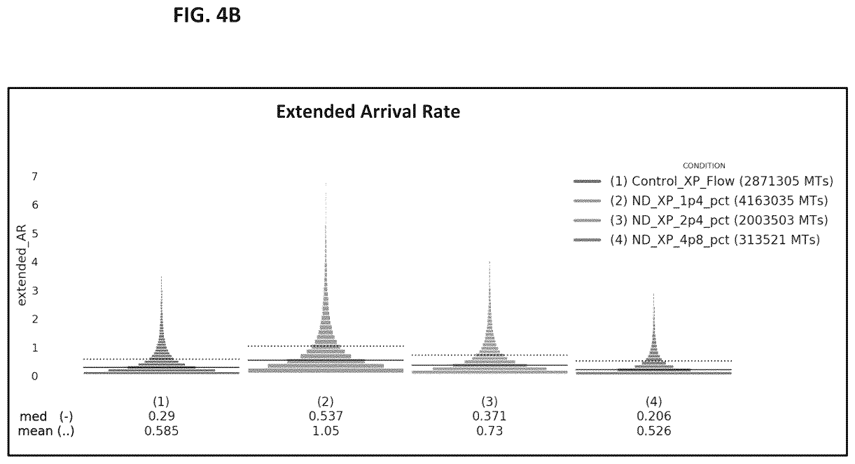

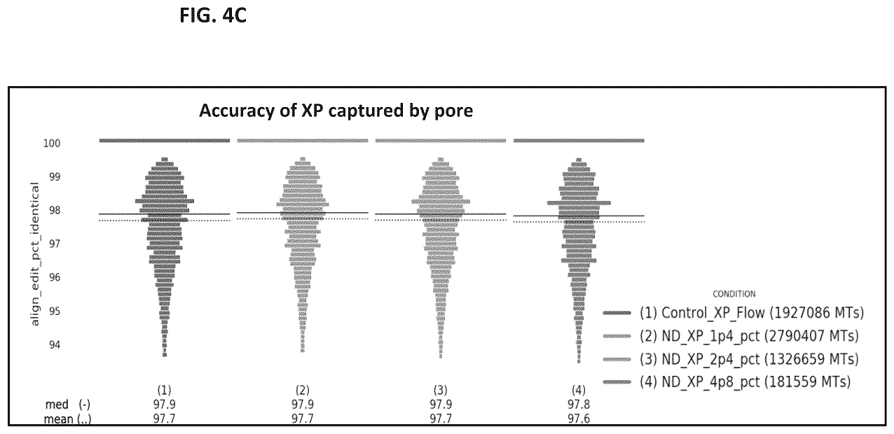

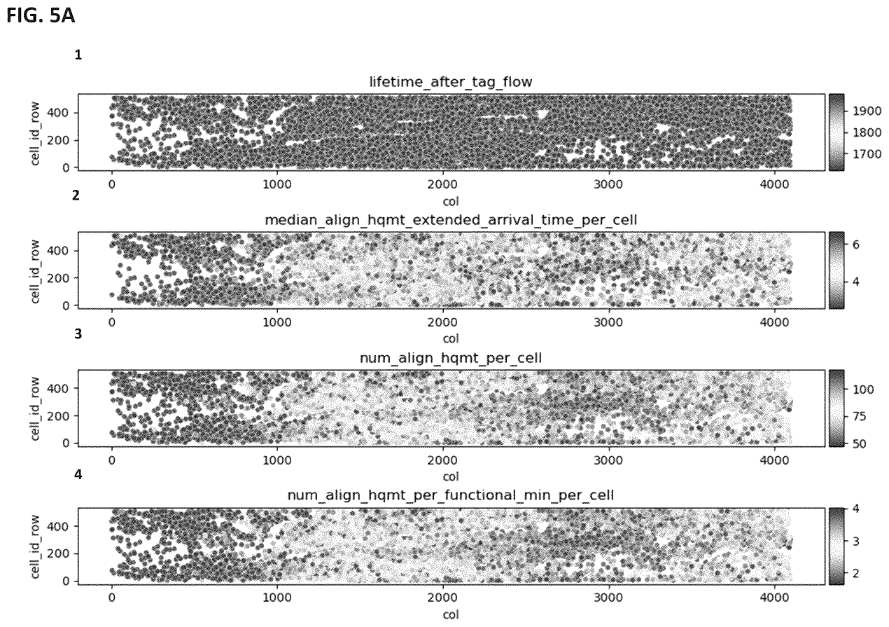

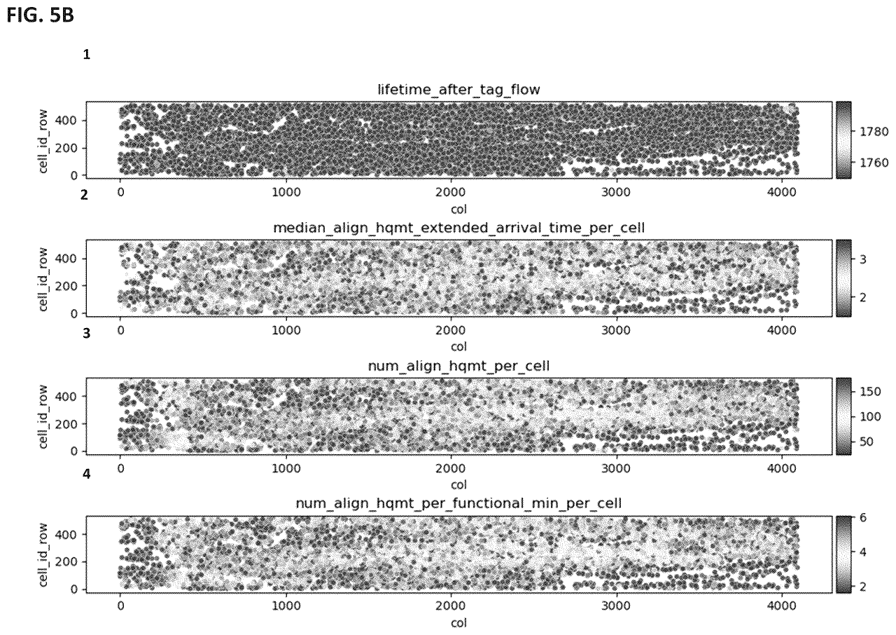

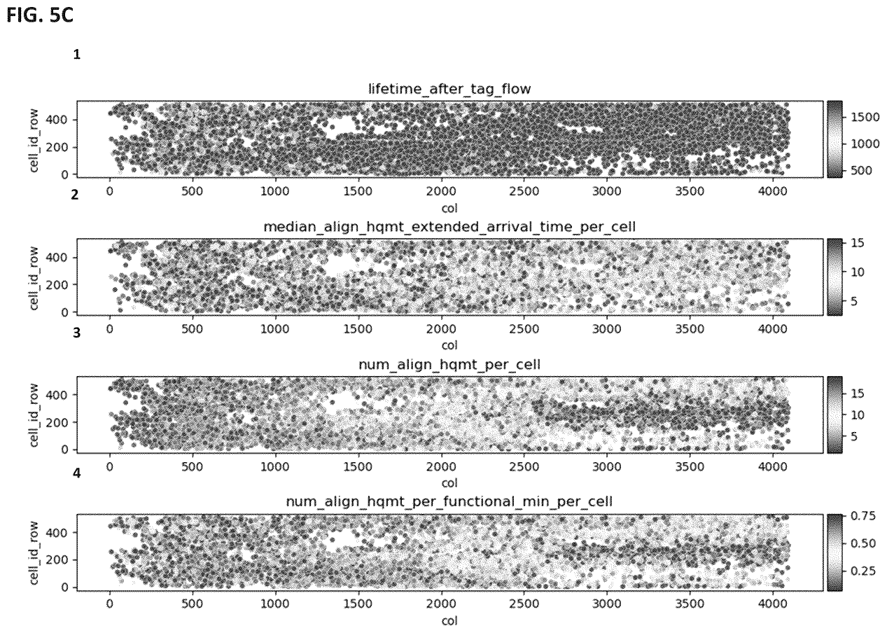

Improved Xpandomer sequencing performance in the presence of mispids is shown in FIGS. 4A-4C and 5A-5C. For example, 1.4% mispid with Xpandomer (orange) gives an almost two-fold throughput improvement over the Xpandomer alone (throughput (1) on the left side, indicated in blue) (FIG. 4A). As for arrival rate, the 1.4% mispid concentration with Xpandomer (throughput (2), indicated in orange) is almost 2× faster over the Xpandomer alone (throughput (1) on the left side, indicated in blue) (FIG. 4B). Yet notably, addition of mispids (1.4%) does not affect sequencing accuracy (FIG. 4C).

With reference to FIGS. 5A-5C, provided are a series of heatmaps showing the spatial distribution of different metrics describing Xpandomer capture along the entirety of a flow cell, in accordance with certain example embodiments. More particularly, FIG. 5A shows four heatmaps (1, 2, 3, and 4) demonstrating capture across the surface of the sequencer flow cell control Xpandomer molecule flow without mispids (i.e., 95% Xpandomer dilution buffer and 5% concentrated Xpandomer). The physical location of each cell of the flow cell is displayed by plotting cell_id_row on the y-axis versus columns on the x-axis. The two bottom heatmaps (3 and 4) depict capture metrics and clearly shows a concentration gradient where Xpandomer is highest (red) on the left side (columns 0-1000) where the inlet is located and decreases (blue) towards the outlet (right).

FIG. 5B shows four heatmaps (1-4) as in FIG. 5A, but with mispids included in the flow solution (i.e., 90.48% Xpandomer dilution buffer, 4.93% concentrated Xpandomer, and 1.41% of a 1:200 MSP2N2:DPhPE mispid preparation). FIG. 5C also shows four heatmaps (1-4), but with a higher concentration of mispids (i.e., 90.48% Xpandomer dilution buffer, 4.76% concentrated Xpandomer, and 4.76% of a 1:200 MSP2N2:DPhPE mispid preparation). As can be seen, the two bottom heatmaps (i.e., heatmaps 3 and 4) of FIG. 5B show a substantial change in capture distribution in the presence of the mispids. Xpandomer capture is also more evenly distributed along the flow cell as demonstrated by the red cells from columns 3000-4000.

Regarding FIG. 5C, again shown are four heatmaps (1-4), but with a higher concentration of mispids (i.e., 90.48% Xpandomer dilution buffer, 4.76% concentrated Xpandomer, and 4.76% of a 1:200 MSP2N2:DPhPE mispid preparation). As can be seen in the bottom two heatmaps (i.e., heatmaps 3 and 4), the capture distribution shifts right when higher levels of mispid are mixed with the Xpandomer. That is, the capture distribution is pushed towards the outlet as demonstrated by the red cells from columns 2500-4000. It is believed, for example, that the higher mispid concentration outcompetes the Xpandomer for space on the bilayer as demonstrated by the low capture cells from column 0-2000 (blue). Yet overall, as can be seen by comparing heatmaps 3 and 4 of FIGS. 5A-5B, addition of 1.41% of the mispid preparation (FIG. 5B) results in more even capture of the Xpandomer molecules across the flow cell versus control (FIG. 5A), while higher amounts of mispid (4.76%, FIG. 5C) results in decreased capture versus control (FIG. 5A).

Sequencing was performed according to the following parameters and settings: Pore: P-0445 loading concentration 0.4 nM, Xpandomer concentration ~1.25 nM, waveform: SBX3a, Sequencing time: 30 min, Labcodes Branch: PEG_6.0.1_SBX, Lacodes Distribution/Release:rel/5.4.2, NSParameters: 6.0.1/SBX_PEG, ACAP: Docker 16 (sbx-dev-ms2-conda-fc6514f) 1% subsampling, Runs, cycles: 4stations X 4cycles, single lane multicycle Xpandomer Manual Mix, N=4. High quality: Xpandomer molecules with greater than 90% sequence accuracy and length greater than 40 nucleotides. Molecular trace (mt): historical notation for SBX signal that indicates Xpandomer nucleotides passing through the pore. num_hqmt_count: number quality (mt); of high molecular trace num_ahqmt_per_functional_min_per_cell: number of high quality molecular traces for a single pore/cell during its observed functional life time; extended_arrival_rate: time between the beginning of an Xpandomer captured by the pore and the time until the next Xpandomer is captured by the same pore.

### Example 3—Target Molecule Sequencing With & Without Lipid Binding Proteins

This example describes a sequencing reaction of a target molecule in the presence of lipid binding proteins, i.e., lipid binding proteins that are not associated with a lipid component to form a mispid. That is, to demonstrate that the improvement to sequencing and capture can be achieved with the lipid binding protein, and to evaluate sequencing metrics of Example 2 without the lipid component, testing was performed as in Example 2, but with only the lipid binding protein, saposin A. Briefly, Xpandomerandomer sequencing was carried out according to Int'l Pat. App. WO2020236526 (see Example 2), without saposin and with varying saposin concentrations 1.25, 2.5, 5, 10 and 20 μM concentration with 1 μL of Xpandomer flow and was tested using the following sequencing conditions: Pore: P-0445 loading concentration 0.4 nM, Xpandomer concentration ~1.25 nM, waveform: SBX3a, Sequencing time: 30 min, Labcodes Branch: PEG_6.0.1_SBX, Lacodes Distribution/Release:rel/5.4.2, NSParameters: 6.0.1/SBX_PEG, ACAP: Docker 16 (sbx-dev-ms2-conda-fc6514f) 1% subsampling, Runs, cycles: 4stations X 4cycles, single lane multicycle Xpandomer Manual Mix, N=4.

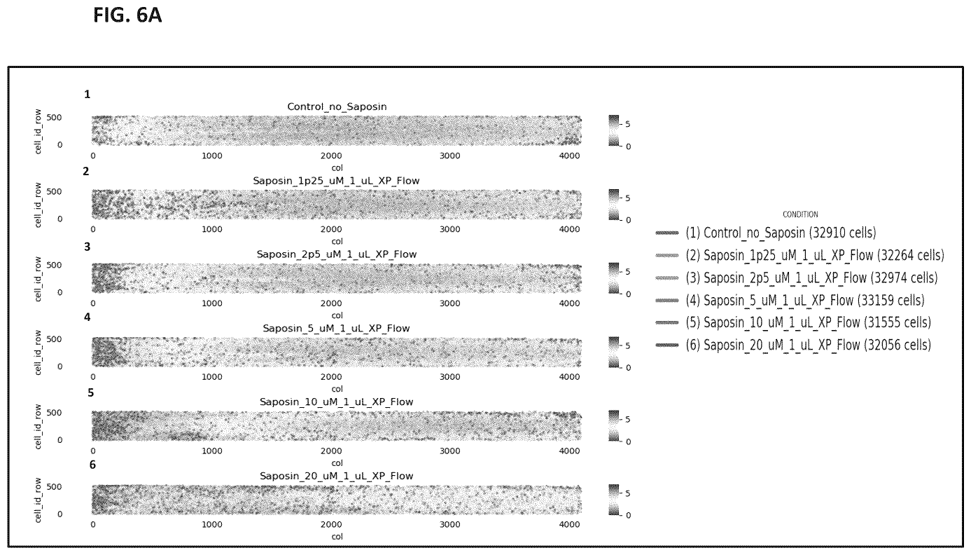

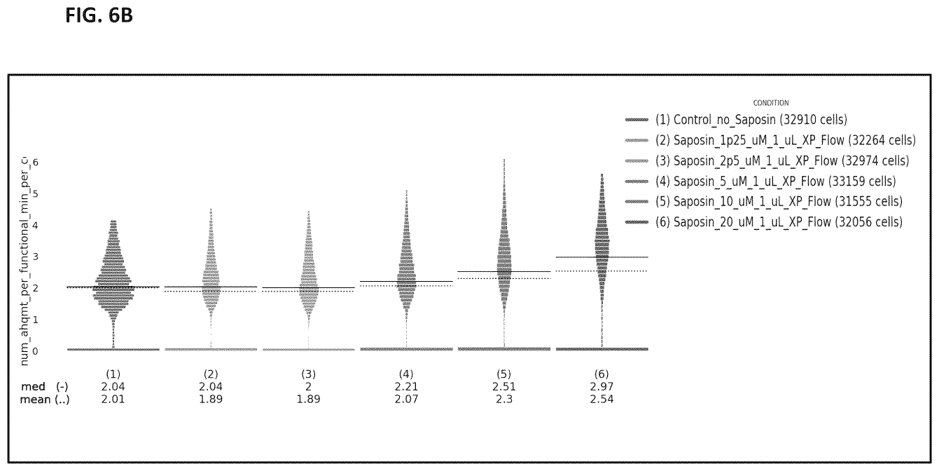

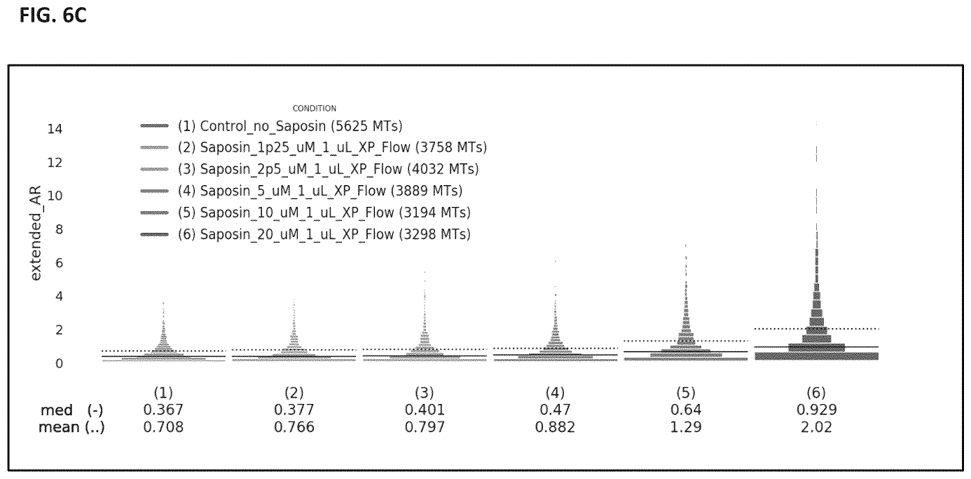

FIGS. 6A-6C include a heatmap (FIG. 6A) showing spatial distribution of Xpandomer molecule capture frequency along the flow cell and histograms (FIGS. 6B-6C) for Xpandomer capture metrics in the presence of increasing concentrations of saposin (lipid binding protein only), in accordance with certain example embodiments. More particularly, FIG. 6A shows the Xpandomer capture distribution along the flow cell from columns 0-4000 when treated with increasing concentrations of saposin with saposin. A concentration of 20 μM performed the best (lane 6) although all conditions performed better than the Xpandomer alone (lane 1). FIG. 6B demonstrates the overall Xpandomer capture for each condition of Xpandomer and saposin with the following metrics detailing the highest improvement of the 20 μM saposin condition of 2.97 compared to the control without saposin 2.04. FIG. 6C demonstrates the overall Xpandomer capture rate for each condition of Xpandomer and increasing concentrations of saposin with the following metrics detailing the highest improvement of the 20 μM saposin condition of 0.929 compared to the control without saposin 0.397. As shown, higher and more even capture distribution is correlated with saposin concentration with the 20 μM concentration demonstrating the highest capture rate and more even flow cell distribution.

### Example 4—Sequencing of Target Molecule (FauXmer)

This example describes a sequencing reaction of a target molecule that is not an Xpandomer. That is, to demonstrate the utility of the mispid to improve capture of molecules that are not the Xpandomer, we used the fauXmer molecule. The fauXmer, for example, includes the concentrator element responsible for causing the Xpandomer molecule to sequester at the bilayer. The fauXmer has the following sequence (SEQ ID NO:1):

| | |
|---|---|
| 5′-LLLLLLLLLLLLLLLLLLLLLLLLLZZZZZZTCATAAGACGAACGGADDDDD-Biotin | L-C2 |
| | Z-C12 |
| | T-dNTP |
| | C-2′MeO dCTP |
| | A-2′MeO dATP |
| | G-2′MeO dGTP |
| | D-PEGG |

In experiments using the FauXmer, the fauXmer is used in place of the Xpandomer and performance metrics focus primarily on capture rates and fauxmer counts as opposed to accuracy and mts (molecular trace).

Briefly, sequencing runs were performed with fauXmer and with varying volumes of mispid (0.5, 1, 2, 4 and 8 μL) spiked into the fauXmer diluted in Xpandomer dilution buffer to 25 nM fauXmer. The samples were tested using the following sequencing conditions and settings: Rev.5 baseline buffers—pH 7.4 HEPES, Waveform: SBX3a (santa clara baseline), Membrane: PE:Span80 lipid, Sequencing time: 30 min, Shorts controller, Xpandomer Lot: FauXmer, ACAP Analysis: fauXmer, 1% subsampling used, 4 stations×6 cycles, N=6. The 0.5 μL mispid spike-in performed the best over the control in metrics used to define yield and efficiency: mt_count of 20 for the fauXmer control and 40 for the 0.5 μL spike-in, throughput of 1.97 for the fauXmer control and 3.78 for the 0.5 μL spike-in. Sequencing conditions and parameters were as described in Examples 1 and 2.

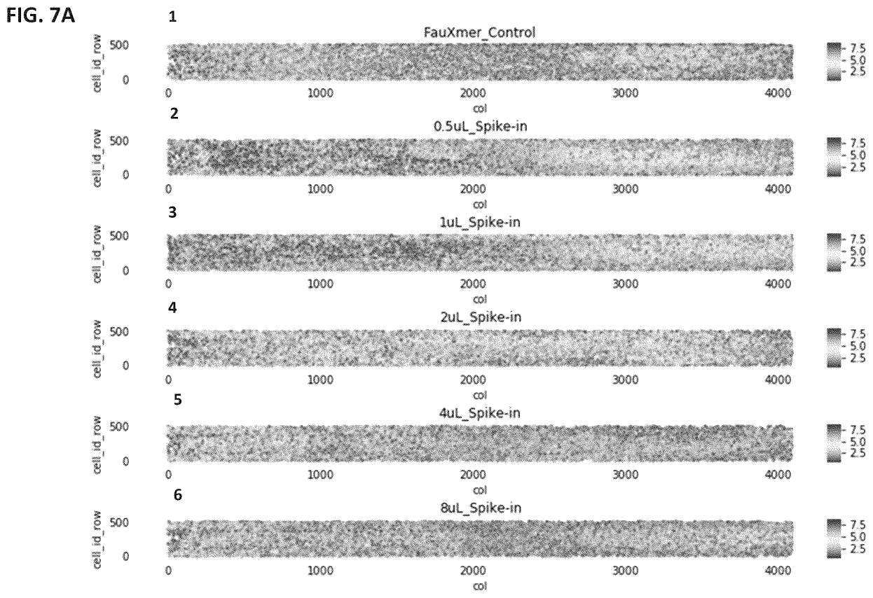

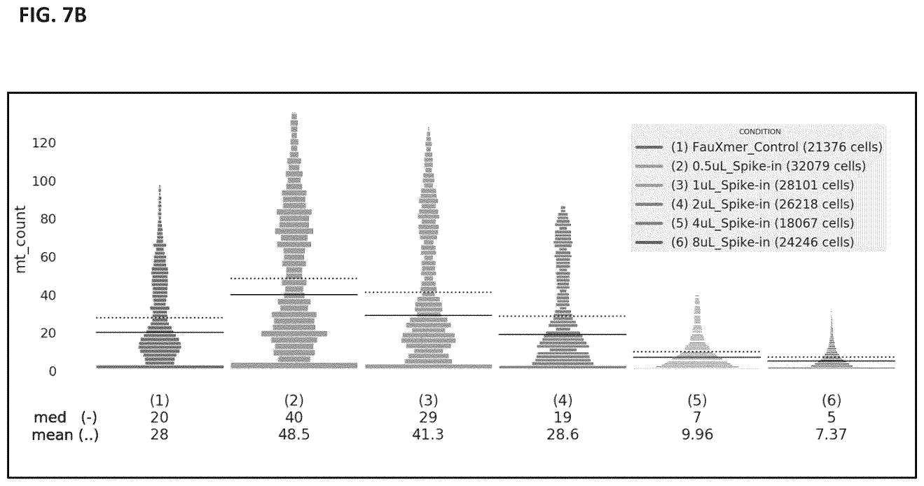

FIGS. 7A-7B include a heatmap (FIG. 7A) showing spatial capture distribution of an alternate molecule, the fauXmer, in the presence of increasing mispid concentration and histograms (FIG. 7B) showing the overall number of fauxmer (mt_count), in accordance with certain example embodiments. More particularly, FIG. 7A shows fauXmer capture heat maps showing the distribution of capture across the flow cell (columns 0-4000) when the fauXmer is mixed with increasing concentrations of mispid (lane 1-6). The highest amount of capture occurs from 0 to 3000 for the 0.5 μL mispid spike-in compared to Capture is distributed from columns 0 to 1000 for the fauXmer control. FIG. 7B provides histograms showing the overall number of fauXmer (mt_count) for the fauXmer control and five conditions where the mispid is mixed in with the fauXmer where the fauXmer count is highest in the 0.5 μL mispid condition of 40 over the fauXmer control without mispid of 20. Mispid drastically improves fauXmer capture distribution across the flow cell. Higher concentrations of mispid reduce fauXmer capture.

### Example 5—Sequencing Reactions Showing Reaction where No Mispids are Flowed Across Cell Followed by Target Molecule

This example describes the experimental set up and results that demonstrate that the mechanism of the mispid/saposin is due to an interaction with the bilayer. A bilayer-mediated mechanism would theoretically have the same improvement in Xpandomer capture if the mispid and the target molecule are not mixed together. To test this theory, these experiments required the use of “two flow” labcodes that allow two consecutive flows where Xpandomer (with or without additives) is flowed in the second flow after an initial flow of other molecules that could impact sequencing. Condition 3, 4 and 5 below are with the two flow labcodes were performed, are compared to the standard single flow experiments (conditions 1 and 2) and are detailed in Table 1.

TABLE 1
Two flow experiment conditions and results (XP = Xpandomer)

| | | | | | Throughput/ |
|---|---|---|---|---|---|
| | | | | | Flow cell |
| | | First | Second | Purpose/ | capture |
| Condition | Test | flow | flow | outcome | distribution |
| 1 | Baseline | none | XP | Standard single | 1.9/0-500 |
| | control | | | flow experiment | |
| 2 | standard | none | XP | Reproduce mispid | 3.8/0-4000 |
| | mispid/XP | | with | effect in standard | |
| | spike-in | | mispid | single flow | |
| | | | | experiment with | |
| | | | | this XP lot | |
| 3 | Two flow | buffer | XP | addition of first | 1.9/0-500 |
| | labcodes | | | flow of buffer | |
| | control | | | does not change | |
| | | | | accuracy or yield | |
| 4 | no spike-in | mispid | XP | improvement in | 2.7/0-1000 |
| | flow rate 1 | | | yield over the | |
| | (0.1 uL/ | | | control is seen; | |
| | second) | | | capture | |
| | | | | distribution from | |
| | | | | 0 to 100 columns | |
| 5 | no spike-in | mispid | XP | improvement in | 2.71/500- |
| | flow rate 2 | | | yield over the | 1000 |
| | (0.01 uL/ | | | control is seen; | |
| | second) | | | capture | |
| | | | | distribution | |
| | | | | shifted from 500 | |
| | | | | to 1000 columns | |

This experiment demonstrates that the mispid effect is primarily due to interaction with the bilayer.

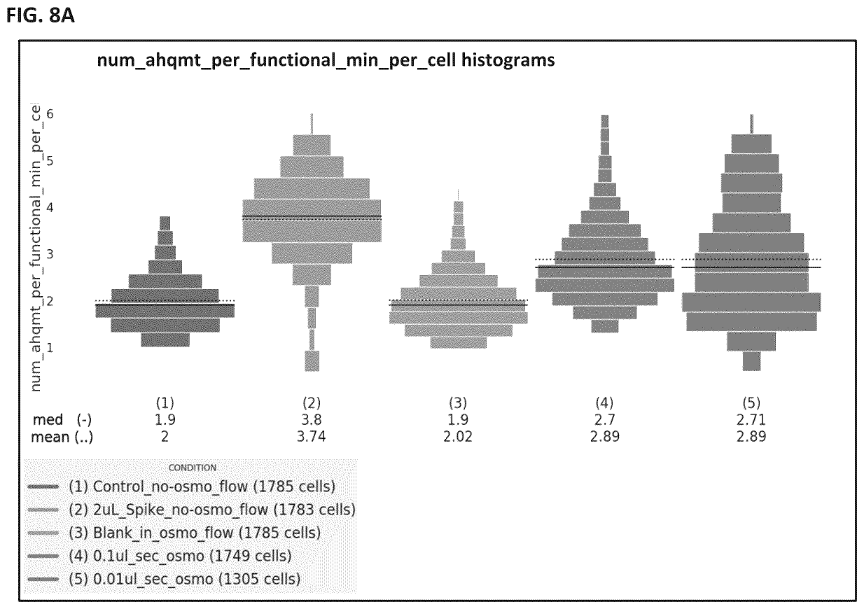

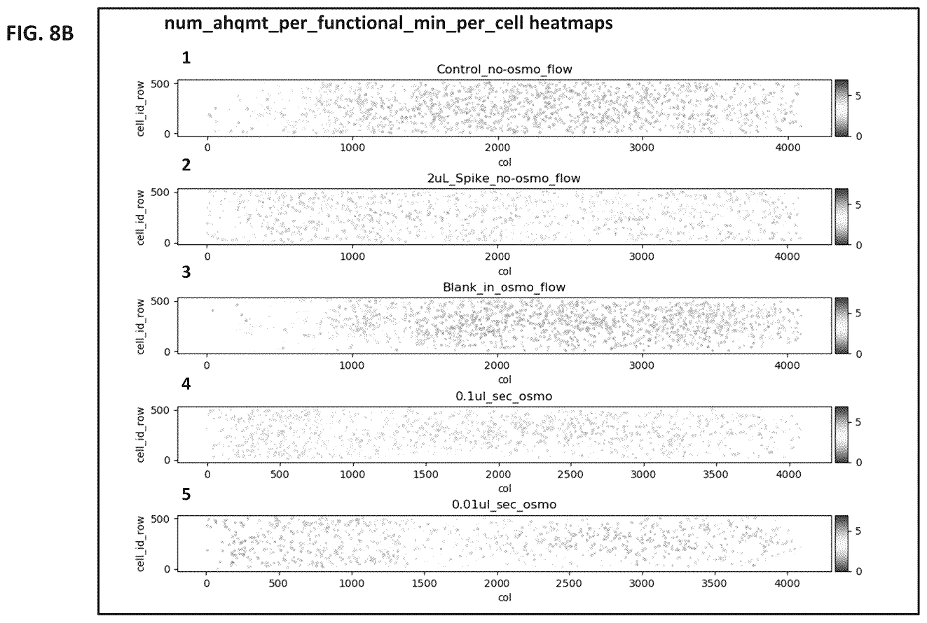

FIGS. 8A-8B include histograms (FIG. 8A) showing the overall number of high quality Xpandomer captures (num_ahqmt_per_functional_min_per_cell) and a heatmap (FIG. 8B) showing Xpandomer capture across the flow cell, in accordance with certain example embodiments. More particularly, FIG. 8A shows histograms showing the overall number of high quality Xpandomer captures (num_ahqmt_per_functional_min_per_cell) for each condition tested where throughput is improved when the mispid is flowed first and the Xpandomer second over the Xpandomer control alone (red and purple histograms compared to the green histogram). As shown, Xpandomer throughput is unchanged with the two flow setup (blue and green) and when the flow rate is modified (red and purple). Throughput improvements over the control are seen in all mispid conditions however, mispid added directly to the Xpandomer continues to perform better than directly modifying the bilayer (orange vs red/green). FIG. 8B demonstrates the Xpandomer capture across the flow cell from column 0-4000 in lanes 1-5 for each condition described in table 1. A comparison of lanes 3, 4 and 5 demonstrates the Xpandomer capture location across the flow cell can be manipulated by changing the flow rate. Heatmaps 1 and 3 in FIG. 8B show Xpandomer capture distribution is unchanged with the two flow setup while heatmaps 4 and 5 show capture distribution is drastically changed as the flow rate is varied, demonstrating the ability to control Xpandomer capture location along the flow cell.

While this experiment clearly demonstrates the bilayer mediated mechanism of the mispid/saposin effect, the throughput improvement where Xpandomer is directly mixed with mispid (FIG. 8A orange histogram and FIG. 8B lane 2) remains the best performing and is subject to ongoing experiments. Sequencing conditions for this experiment was as follows: Rev.5 Baseline Buffer: pH 7.4 HEPES, Waveform: SBX3a (Santa Clara Baseline), Sequencing time: 30 min, Shorts controller, Xpandomer lot: PXPC00043, ACAP Analysis: Docker 19, 1% subsampling used, 4 stations×5 cycles.

In view of the many possible embodiments to which the principles of the disclosed invention may be applied, it should be recognized that the illustrated example embodiments are only preferred examples of the invention and should not be taken as limiting the scope of the invention. Rather, the scope of the invention is defined by the following claims. We therefore claim as our invention all that comes within the scope and spirit of these claims.

1-54. (canceled)

55. A method for sequencing a target molecule, comprising:
providing a chip, the chip comprising a plurality of sensing electrodes and a lipid membrane that is disposed adjacent to or in proximity to the sensing electrodes, wherein a plurality of nanopore assemblies are disposed within the lipid membrane;
contacting the chip with a plurality of lipid binding proteins;
contacting the chip with a target molecule;
applying a voltage across the membrane;
determining, by one or more of the sensing electrodes, one or more current or voltage changes associated with the nanopore assembly; and
determining, with the aid of a computer processor and based on the one or more of the determined current or voltage changes associated with the nanopore assembly, a sequence for the target molecule,
wherein at least a portion of the plurality of lipid binding proteins are complexed with a lipid component to form a plurality of mispids and wherein the chip is contacted with the plurality of mispids, thereby contacting the chip with a plurality of lipid binding proteins.

56. The method of claim 55, wherein the target molecule is a nucleic acid, a modified nucleic acid, or a nucleic acid surrogate.

57. The method of claim 56 wherein the nucleic acid surrogate comprises an Xpandomer.

58. The method of claim 55, wherein the plurality of mispids is prepared by contacting a plurality of lipid binding proteins with a lipid component, thereby forming the plurality of mispids, and wherein the lipid binding protein is a membrane scaffolding protein (MSP).

59. The method of claim 58, wherein the MSP is an apolipoprotein or derivative thereof.

60. The method of claim 55, wherein the lipid binding protein comprises 3-10 alpha helices.

61. The method of claim 55, wherein the ratio of the lipid binding protein to the lipid component is approximately 1:200 to 1:1500.

62. The method of claim 55, wherein the ratio of the lipid binding protein to the lipid component is approximately 1:1200.

63. The method of claim 55, wherein the lipid component comprises a phosphatidylcholine-based lipid, a phosphoethanolamine-based lipid, a derivative thereof, or a combination thereof.

64. The method of claim 63, wherein the lipid component comprises 1,2-Diphytanoyl-sn-Glycero-3-Phosphatidylcholine (DPhPC), 1,2-diphytanoyl20 sn-glycero-3-phosphoethanolamine (DphPE), derivatives thereof, or combinations thereof.

65. The method of claim 55, wherein the mispids and target molecules are mixed together before contacting the lipid membrane.

66. A composition for modifying the interaction of a target molecule with a lipid membrane, the composition comprising a heterogenous mixture of a lipid component and lipid binding proteins.

67. The composition of claim 66, wherein the lipid binding protein is a membrane scaffolding protein (MSP).

68. The composition of claim 67, wherein the MSP is an apolipoprotein or derivative thereof.

69. The composition of claim66, wherein the lipid binding protein comprises 3-10 alpha helices.

70. The composition of claim 66, wherein the ratio of the lipid binding protein to the lipid component is approximately 1:600 to 1:1500.

71. The composition of claim 66, wherein the ratio of the lipid binding protein to the lipid component is approximately 1:1200.

72. The composition of claim 66, wherein the ratio of the lipid binding protein to the lipid component is approximately 1:200.

73. The composition of claim 66, wherein the lipid component comprises a phosphatidylcholine-based lipid, a phosphoethanolamine-based lipid, a derivative thereof, or a combination thereof.

74. The composition of claim 73, wherein the lipid component comprises 1,2-Diphytanoyl-sn-Glycero-3-Phosphatidylcholine (DPhPC), 1,2-diphytanoylsn-glycero-3-phosphoethanolamine (DphPE), derivatives thereof, or combinations thereof.


SEQUENCE LISTING (1 sequence)

SEQ ID NO: 1 - DNA, 16 bp, synthetic construct
  source 1..16: mol_type = other DNA
  modified_base 2: mod_base = cm
  modified_base 9: mod_base = cm
  modified_base 13: mod_base = cm
  modified_base 7: mod_base = gm
  modified_base 10: mod_base = gm
  modified_base 14..15: mod_base = gm
  modified_base 3: mod_base = OTHER; note = 2'-O-methyladenosine
  modified_base 5..6: mod_base = OTHER; note = 2'-O-methyladenosine
  modified_base 8: mod_base = OTHER; note = 2'-O-methyladenosine
  modified_base 11..12: mod_base = OTHER; note = 2'-O-methyladenosine
  modified_base 16: mod_base = OTHER; note = 2'-O-methyladenosine
tcataagacgaacgga